(19) United States
(12) Patent Application Publication
(10) Pub. No.: US 2014/0323477 A1
(43) Pub. Date: Oct. 30, 2014

(54) SERINE/THREONINE KINASE INHIBITORS

(71) Applicant: Genentech, Inc., South San Francisco, CA (US)

(72) Inventors: James J. CRAWFORD, San Francisco, CA (US); Wendy LEE, San Ramon, CA (US); Joachim RUDOLPH, Burlingame, CA (US)

(73) Assignee: GENENTECH, INC., South San Francisco, CA (US)

(21) Appl. No.: 14/266,617

(22) Filed: Apr. 30, 2014

Related U.S. Application Data
(60) Provisional application No. 61/817,530, filed on Apr. 30, 2013.

(51) Int. Cl. C07D 471/04; A61K 45/06; A61K 31/5377; A61K 31/519

(52) U.S. Cl. CPC C07D 471/04; A61K 31/519; A61K 45/06; A61K 31/5377
USPC 514/234.2; 544/279; 514/264.11; 544/117

(57) ABSTRACT

Compounds having the formula I, or a pharmaceutically acceptable salt thereof, wherein R¹, R², X¹, X², and Ar are as defined herein, are inhibitors of PAK1. Also disclosed are compositions and methods for treating cancer and hyperproliferative disorders using such compounds.

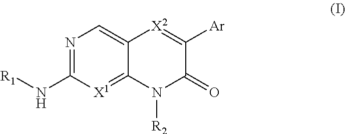

### CROSS-REFERENCE TO PRIOR APPLICATIONS

This application claims the benefit of priority to U.S. Ser. No. 61/817,530, filed Apr. 30, 2013, which is incorporated herein by reference in its entirety.

### FIELD OF THE INVENTION

The present invention relates to compounds which inhibit serine/threonine kinases and which are useful for treating hyperproliferative and neoplastic diseases by inhibiting signal transduction pathways which commonly are overactive or over-expressed in cancerous tissue. The present compounds are inhibitors of group 1 p21-activated protein kinases (PAK1, PAK2, and PAK3). The present invention further relates to methods for treating cancer or hyperproliferative diseases with compounds within the scope of the present invention.

### BACKGROUND OF THE INVENTION

Protein kinases are a family of enzymes that catalyze phosphorylation of the hydroxyl groups of specific tyrosine, serine, or threonine residues in proteins. Typically, such phosphorylation can dramatically change the function of the protein and thus protein kinases can be pivotal in the regulation of a wide variety of cellular process, including metabolism, cell proliferation, cell differentiation, and cell survival. The mechanism of these cellular processes provides a basis for targeting protein kinases to treat disease conditions resulting from or involving a disorder of these cellular processes. Examples of such diseases include, but are not limited to, cancer and diabetes.

Protein kinases can be broken into two types, protein tyrosine kinases (PTKs) and serine-threonine kinases (STKs). Both PTKs and STKs can be receptor protein kinases or non-receptor protein kinases. PAK is a family of non-receptor STKs. The p21-activated protein kinase (PAK) family of serine/threonine protein kinases plays important roles in cytoskeletal organization, cellular morphogenesis, cellular processes and cell survival (Daniels et al., Trends Biochem. Sci. 1999 24: 350-355; Sells et al., Trends Cell. Biol. 1997 7:162-167). The PAK family consists of six members subdivided into two groups: PAK 1-3 (group I) and PAK 4-6 (group II) which are distinguished based upon sequence homologies and the presence of an autoinhibitory region in group I PAKs. p21-Activated kinases (PAKs) serve as important mediators of Rac and Cdc42 GTPase function as well as pathways required for Ras-driven tumorigenesis. (Manser et al., Nature 1994 367:40-46; B. Dummler et al., Cancer Metathesis Rev. 2009 28:51-63; R. Kumar et al., Nature Rev. Cancer 2006 6:459-473).

Changes in the levels and activities of group 1 PAKs in particular, are frequently associated with human malignancies including, but not limited to bladder carcinoma, breast carcinoma, colorectal carcinoma, gastric carcinoma, glioblastoma, hepatocellular carcinoma, ovarian carcinoma and renal cell carcinoma, primary breast adenocarcinoma, squamous non-small cell lung cancer or a squamous head and necks cancer. (J. V. Kichina et al., Expert. Opin. Ther. Targets 2010, 14(7), 703.) PAK1 genomic amplification at 11q13 was prevalent in luminal breast cancer, and PAK1 protein expression was associated with lymph node metastasis. High expression of PAK2 in mammary invasive ductal carcinomas has been associated with increased survival and resistance of breast tumor cells to chemotherapeutic agents (X. Li et al., J. Biol. Chem 2011, 286(25), 2291). Squamous non-small cell lung carcinomas (NSCLCs) and head and neck squamous carcinomas have aberrant cytoplasmic expression of PAK1. (C. C. Ong et al., Proc. Nat. Acad. Sci., USA 2011, 108(17), 7177.) Group 1 PAKs contribute to squamous NSCLC cell motility, survival and proliferation (C. C. Ong et al., Oncotarget 2011 2(6):491) and PAK2 has been linked to mitosis completion in response to various cell stimuli (M. R. Banko et al., Mol. Cell 2011, 44(6), 878-92).

### SUMMARY OF THE INVENTION

There is a continuing need for new and novel therapeutic agents that can be used for cancer and hyperproliferative conditions. The PAK kinases are important signaling proteins frequently over-expressed and/or overactive in many cancerous tissues. Design and development of new pharmaceutical compounds that inhibit or modulate their activity is essential. In one aspect of the present invention there is provided a compound according to formula IA:

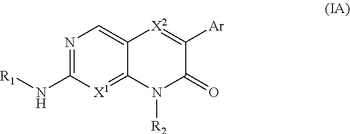

wherein:
X¹ and X² are independently CH or N;
Ar is phenyl, pyridinyl, pyridine-N-oxide, pyridinone, pyrimidinyl, pyridazinyl, or pyrazinyl, each substituted by —S(═O)ₙR³, and optionally further substituted by one or two groups independently selected from C₁₋₆ alkyl, C₁₋₆ alkoxy, C₁₋₆ haloalkyl, C₃₋₆ cycloalkyl, halogen, and cyano; wherein n is 0, 1, or 2; and
   R³ is NR^{3a}R^{3b};
      wherein R^{3a} and R^{3b} are independently selected from:
      (a) hydrogen;
      (b) C₁₋₁₀ alkyl;
      (c) C₁₋₆ haloalkyl; and
      (d) C₃₋₆ cycloalkyl;
         wherein any said alkyl or said cycloalkyl is optionally substituted either with one or two hydroxyl groups or with a NR^{3c}R^{3d} group;
            wherein R^{3a} and R^{3d} are independently hydrogen or C₁₋₃ alkyl; or R^{3c} and R^{3d} together with the nitrogen to which they are attached form a cyclic amine; or
      (e) R^{3a} and R^{3b} together with the nitrogen atom to which they are attached form a cyclic amine, optionally substituted with one or two groups selected from C₁₋₄ alkyl and oxo;
R¹ is (alkylene)₀₋₆R^{1a};
   wherein R^{1a} is (i) hydrogen, (ii) hydroxyl, (iii) C₁₋₆ alkoxy, (iv) NR^{1b}R^{1c}, or (v) a heterocycle selected from the group consisting of azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, N—C₁₋₆ alkyl piperazinyl, tetrahydropyranyl, tetrahydrofuranyl, 3-azabicyclo[3.1.0]hexan-6-yl, and 5-amino-1,3-dioxolan-2-yl, each said heterocycle optionally substituted by oxo, hydroxyl, amino, C₁₋₃ alkylamino, or C₁₋₃ dialkylamino;
      wherein R^{1b} and R^{1c} are independently hydrogen or C₁₋₃ alkyl optionally substituted by OH or NR^{1d}R^{1e}; or R^{1b} and R^{1c} together with the nitrogen to which they are attached form a cyclic amine optionally substituted either with one or two hydroxyl groups or with a NR^{3c}R^{3d} group; wherein R^{1d} and R^{1e} are independently hydrogen or C₁₋₃ alkyl; and
      with the proviso that when R^{1a} is NR^{1b}R^{1c} or OH, the alkylene moiety of R¹ contains at least contains two carbons;
R² is selected from the group consisting of:
   (i) hydrogen;
   (ii) C₁₋₆ alkyl;
   (iii) C₃₋₇ cycloalkyl;
   (iv) (alkylene)₁₋₃NR^{a}R^{b}, wherein the alkylene chain is optionally substituted by a hydroxyl;
   (v) (alkylene)₁₋₃OR⁵, wherein R⁵ is (alkylene)₂₋₄NR^{a}R^{b} or a heterocycle selected from azetidine, pyrrolidine, piperidine, and azepane;
   (vi) (alkylene)₀₋₃-(C₄₋₆-cycloalkyl-NR^{a}R^{b});
   (vii) (alkylene)₀₋₃-heterocyclyl, wherein heterocyclyl refers to azetidinyl, pyrrolidinyl, piperidinyl, azepanyl, morpholinyl, 1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl, 5,8-dioxa-2-azaspiro[3.4]octan-6-yl, 5,5-dioxido-5-thia-2-azaspiro[3.4]octan-7-yl, thiomorpholinyl, or piperazinyl, each optionally substituted by one or more moieties selected from the group consisting of C₁₋₆ alkyl, C(═O)CHR^{f}NH₂, halogen, oxo, hydroxyl, amino, C₁₋₃ alkylamino, C₁₋₃ dialkylamino, and C₁₋₆-hydroxyalkyl;
   wherein R^{a} and R^{b} are independently hydrogen, C₁₋₃ alkyl, or C₂₋₄ hydroxyalkyl; or R^{a} and R^{b} together with the nitrogen to which they are attached form a cyclic amine optionally substituted either with one or two hydroxyl groups or with a NR^{3c}R^{3d} group; and
   R^{f} is hydrogen or C₁₋₃ alkyl;
      or a pharmaceutically acceptable salt thereof.

In another embodiment, there is provided a compound of formula (IB):

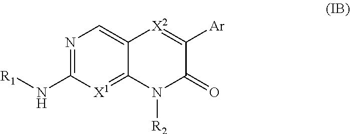

wherein
X is CH or N;
Ar is phenyl, pyridinyl, pyridinyl-N-oxide, pyridone, pyrimidinyl, pyridazinyl or pyrazinyl each substituted by —S(═O)ₙR³ and optionally further substituted by one or two groups independently selected from C₁₋₆ alkyl, C₁₋₆ alkoxy, C₁₋₆ haloalkyl, C₃₋₆ cycloalkyl, halogen, and cyano; wherein n is 1 or 2;
R³ is NR^{3a}R^{3b};
   wherein R^{3a} and R^{3b} are each independently selected from:
   (a) hydrogen;
   (b) C₁₋₁₀ alkyl;
   (c) C₁₋₆ haloalkyl; and
   (d) C₃₋₆ cycloalkyl;
      wherein any said alkyl or said cycloalkyl is optionally substituted either with one or two hydroxyl groups or with a NR^{3c}R^{3d} group;
         wherein R^{3c} and R^{3d} are each independently hydrogen or C₁₋₃ alkyl, or R^{1c} and R^{3d} together with the nitrogen atom to which they are attached form a cyclic amine, or
   (e) R^{3a} and R^{3b} together with the nitrogen atom to which they are attached form a cyclic amine;
R¹ is (alkylene)₀₋₆R^{1a};
   wherein R^{1a} is hydroxyl, C₁₋₆ alkoxy, NR^{1a}R^{1b}, or a heterocycle selected from the group consisting of azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, tetrahydropyranyl, and tetrahydrofuranyl, said heterocycle optionally substituted by oxo, hydroxyl, C₁₋₃ alkylamino, or C₁₋₃ dialkylamino;
      wherein R^{1a} and R^{1b} are independently hydrogen or C₁₋₃ alkyl, or R^{1a} and R^{1b} together with the nitrogen atom to which they are attached form a cyclic amine;
      with the proviso when R^{1a} is NR^{1a}R^{1b} or OH the alkylene moiety of R¹ has at least contains two carbons; and
R² is hydrogen, C₁₋₆ alkyl, or C₃₋₇ cycloalkyl;
   or a pharmaceutically acceptable salt thereof.

Another aspect of the present invention relates to a method for treating a hyperproliferative disorder by administering a therapeutically effective quantity of a compound according to (a) formula IA or (b) formula IB, or a pharmaceutically acceptable salt thereof, to a patient in need thereof. The compound can be administered alone or co-administered with at least one other anti-hyperproliferative or chemotherapeutic compound.

Another aspect of the present invention relates to a method for inhibiting PAK activity in a cell comprising treating a cell with a compound according to (a) formula IA or (b) formula IB, or a pharmaceutically acceptable salt thereof, in an amount effective to attenuate or eliminate PAK activity.

Another aspect of the present invention relates to pharmaceutical compositions containing a compound of (a) formula IA or (b) formula IB, or a pharmaceutically acceptable salt thereof, with one or more pharmaceutically acceptable excipients, diluents and/or carriers.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, by way of example, in a compound in which R″ appears twice and is defined as “independently carbon or nitrogen,” both R″ variables can be carbon, both R″ variables can be nitrogen, or one R″ can be carbon and the other nitrogen.

When any variable (e.g., R¹, R⁴, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

A wavy line “ [symbol] ” drawn through a bond indicates the point of attachment of a functional group or other chemical moiety to the rest of the molecule of which it is a part. Thus, for example:

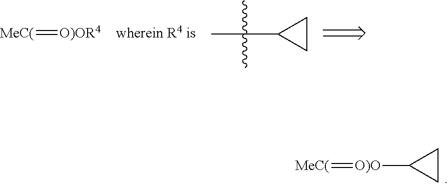

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1, or 2 for variables which are inherently discrete, and can be 0.0, 0.1, 0.01, 0.001, or any other real value for variables which are inherently continuous.

Formula I as used herein refers collectively to compounds of formulae IA and/or IB.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomer usually produces a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH- [symbol] -C(—OH)═CH—), amide/imidic acid (—C(═O)—NH- [symbol] -C(—OH)═N—), and amidine (—C(═NR)—NH- [symbol] -C(—NHR)═N—) tautomers. The latter two are particularly common in heteroaryl and heterocyclic rings and the present invention encompasses all tautomeric forms of the compounds.

It will be appreciated by the skilled artisan that some of the compounds of formula I may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the skilled artisan that substitution of the tropane ring can be in either endo- or exo-configuration, and the present invention covers both configurations. The present invention includes all the individual stereoisomers (e.g., enantiomers), racemic mixtures or partially resolved mixtures of the compounds of formula I and, where appropriate, the individual tautomeric forms thereof.

The compounds of formula I may contain an acidic or basic center and suitable salts are formed from acids or bases may form non-toxic salts which have similar biological activity. Examples of salts of inorganic acids include the hydrochloride, hydrobromide, hydroiodide, chloride, bromide, iodide, sulfate, bisulfate, nitrate, phosphate, and hydrogen phosphate salts. Examples of salts of organic acids include acetate, fumarate, pamoate, aspartate, besylate, carbonate, bicarbonate, camsylate, D and L-lactate, D and L-tartrate, esylate, mesylate, malonate, orotate, gluceptate, methylsulfate, stearate, glucuronate, 2-napsylate, tosylate, hibenzate, nicotinate, isethionate, malate, maleate, citrate, gluconate, succinate, saccharate, benzoate, esylate, and pamoate salts. For a review on suitable salts, see Berge et al, J. Pharm. Sci., 1977 66:1-19 and G. S. Paulekuhn et al. J. Med. Chem. 2007 50:6665.

In one embodiment of the present invention there is provided a compound of formula IA or IB wherein X¹ and X² (may be referred to collectively as X) are independently CH or N. In some embodiments, X¹ is N and X² is CH.

In another embodiment, there is provided a compound of formula IA, wherein:
X¹ and X² are independently CH or N;
Ar is phenyl, pyridinyl, pyridine-N-oxide, pyridinone, pyrimidinyl, pyridazinyl, or pyrazinyl, each substituted by —S(═O)—R³, and optionally further substituted by one or two groups independently selected from C₁₋₆ alkyl, C₁₋₆ alkoxy, C₁₋₆ haloalkyl, C₃₋₆ cycloalkyl, halogen, and cyano; wherein n is 0, 1, or 2; and
   R³ is NR^{3a}R^{3b};
      wherein R^{3a} and R^{3b} are independently selected from:
      (a) hydrogen;
      (b) C₁₋₁₀ alkyl;
      (c) C₁₋₆ haloalkyl; and
      (d) C₃₋₆ cycloalkyl;
         wherein any said alkyl or said cycloalkyl is optionally substituted either with one or two hydroxyl groups or with a NR^{3c}R^{3d} group;
            wherein R^{3c} and R^{3d} are independently hydrogen or C₁₋₃ alkyl; or R^{1c} and R^{3d} together with the nitrogen atom to which they are attached form a cyclic amine; or
      (e) R^{3a} and R^{3b} together with the nitrogen atom to which they are attached form a cyclic amine, optionally substituted with one or two groups selected from C₁₋₄ alkyl and oxo;
R¹ is (alkylene)₀₋₆R^{1a};
   wherein R^{1a} is hydroxyl, C₁₋₆ alkoxy, NR^{1b}R^{1c}, or a heterocycle selected from the group consisting of azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, N—C₁₋₆ alkyl piperazinyl, tetrahydropyranyl, tetrahydrofuranyl, 3-azabicyclo[3.1.0]hexan-6-yl, and 1,3-dioxolan-5-yl, each said heterocycle optionally substituted by oxo, hydroxyl, amino, C₁₋₃ alkylamino, or C₁₋₃ dialkylamino;
      wherein R^{1b} and R^{1c} are independently hydrogen or C₁₋₃ alkyl optionally substituted by a OH or NR^{1d}R^{1e}; or R^{1b} and R^{1c} together with the nitrogen atom to which they are attached form a cyclic amine;
         wherein R^{1d} and R^{1e} are independently hydrogen or C₁₋₃ alkyl; and
      with the proviso that when R^{1a} is NR^{1b}R^{1c} or OH, the alkylene moiety of R¹ contains at least contains two carbons;
R² is hydrogen, C₁₋₆ alkyl, or C₃₋₇ cycloalkyl;
   or a pharmaceutically acceptable salt thereof.

In one embodiment of the present invention there is provided a compound according to formula IB as defined hereinabove. In one embodiment of the present invention there is provided a compound according to formula IB wherein X¹, X², Ar, R¹, R², R³, R⁴, R^{1a}, R^{1b}, R^{1c}, R^{1d}, R^{3a}, R^{3b}, R^{3c}, R^{3d} and n are as defined hereinabove. In other embodiments, there is provided a compound of formula IA as defined hereinabove.

In another embodiment of the present invention there is provided a compound of formula IA wherein X¹ is N, X² is CH, Ar is phenyl, and all other variables are defined as hereinabove. In another embodiment of the present invention there is provided a compound of formula IB wherein X¹ is N, X² is CH, Ar is phenyl, and all other variables are defined as hereinabove. In such embodiments, Ar is phenyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ and X² are CH, Ar is phenyl, and all other variables are defined as hereinabove. In another embodiment of the present invention there is provided a compound of formula IB wherein X¹ and X² are CH, Ar is phenyl, and all other variables are defined as hereinabove. In such embodiments, Ar is phenyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ and X² are N, Ar is phenyl, and all other variables are defined as hereinabove. In another embodiment of the present invention there is provided a compound of formula IB wherein X¹ and X² are N, Ar is phenyl, and all other variables are defined as hereinabove. In such embodiments, Ar is phenyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ is N, X² is CH, Ar is pyridinyl, and all other variables are defined as hereinabove. In another embodiment of the present invention there is provided a compound of formula IB wherein X¹ is N, X² is CH, Ar is pyridinyl, and all other variables are defined as hereinabove. In such embodiments, Ar is pyridinyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ and X² are CH, Ar is pyridinyl, and all other variables are defined as hereinabove. In another embodiment of the present invention there is provided a compound of formula IB wherein X¹ and X² are CH, Ar is pyridinyl, and all other variables are defined as hereinabove. In such embodiments, Ar is pyridinyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ and X² are N, Ar is pyridinyl, and all other variables are defined as hereinabove. In another embodiment of the present invention there is provided a compound of formula IB wherein X¹ and X² are N, Ar is pyridinyl, and all other variables are defined as hereinabove. In such embodiments, Ar is pyridinyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ is N, X² is CH, Ar is pyrimidinyl, and all other variables are defined as hereinabove. In another embodiment of the present invention there is provided a compound of formula IB wherein X¹ is N, X² is CH, Ar is pyrimidinyl, and all other variables are defined as hereinabove. In such embodiments, Ar is pyrimidinyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In another embodiment of the present invention there is provided a compound of formula IA wherein X¹ is N, X² is CH, Ar is pyrimidinyl, R³ is NHC₁₋₆ alkyl or N(C₁₋₆ alkyl)₂, and all other variables are defined as hereinabove. In another embodiment of the present invention there is provided a compound of formula IB wherein X¹ is N, X² is CH, Ar is pyrimidinyl, R³ is NHC₁₋₆ alkyl or N(C₁₋₆ alkyl)₂, and all other variables are defined as hereinabove. In such embodiments, Ar is pyrimidinyl substituted by —S(═O)₀₋₂R³, and optionally further substituted by one or two groups independently selected from C₁₋₆ alkyl, C₁₋₆ alkoxy, C₁₋₆ haloalkyl, C₃₋₆ cycloalkyl, halogen, and cyano.

In some embodiments, Ar is phenyl, pyridinyl, or pyrimidinyl, each substituted by —S(═O)—R³, and optionally further substituted by one or two groups independently selected from C₁₋₆ alkyl, C₁₋₆ alkoxy, C₁₋₆ haloalkyl, C₃₋₆ cycloalkyl, halogen, and cyano. In other embodiments, Ar is phenyl or pyridinyl, each substituted by —S(═O)—R³, and optionally further substituted with C₁₋₄ alkyl or halogen. In other embodiments, Ar is phenyl substituted with —S(═O)—R³, and optionally further substituted with C₁₋₄ alkyl or halogen. In some embodiments, Ar is IIa, or is IIb, IIc, or IId:

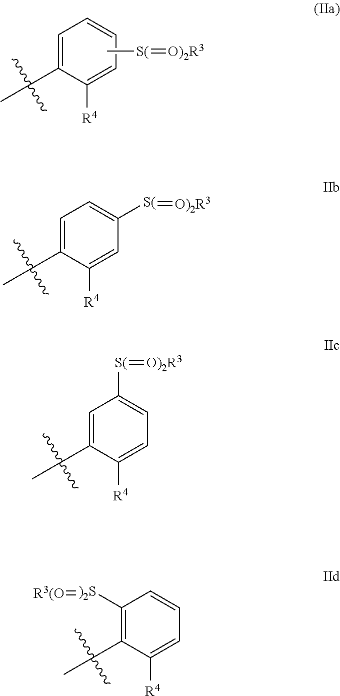

wherein R⁴ is C₁₋₃ alkyl, bromo, chloro, fluoro, cyano, OMe, or CF₃. In some embodiments, Ar is IIa. In other embodiments, Ar is IIb. In other embodiments, Ar is IIc. In other embodiments, Ar is IId. In some embodiments of each of IIa, IIb, IIc, and IId, R⁴ is methyl or chloro.

In another embodiment of the present invention there is provided a compound of (a) formula IA or (b) formula IB, wherein X¹ is N; X² is CH; Ar is IIa, wherein R⁴ is C₁₋₃ alkyl, bromo, chloro, fluoro, cyano, OMe or CF₃; R¹ is selected from Scheme 1; and, R^{3a} and R^{3b} are each independently hydrogen or optionally substituted C₁₋₆ alkyl, or R^{3a} and R^{3b} together with the nitrogen to which they are attached form pyrrolidine, piperidine, morpholine, piperazine, N-methyl-piperazine, or 1,1-dioxo-thiomorpholine, and all other variables are as defined hereinabove.

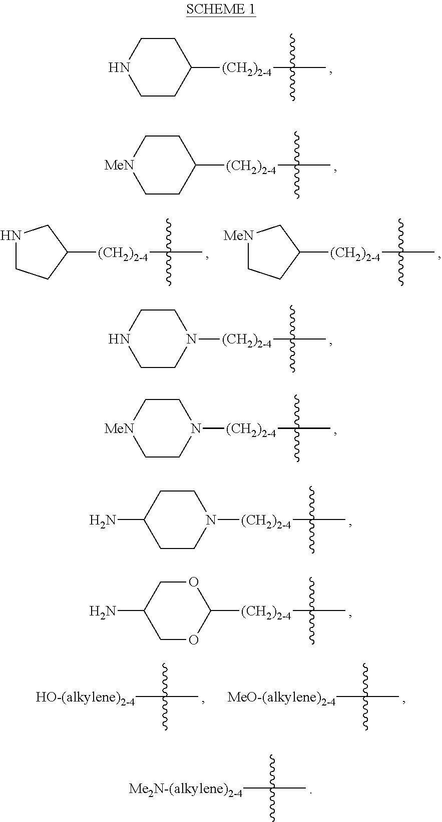

In another embodiment of the present invention there is provided a compound of formula IA or IB, wherein X¹ is N, X² is CH, Ar is IIa, R¹ is C₁₋₃ alkyl, R² is (alkylene)₁₋₂-heterocyclyl, and heterocyclyl is selected from Scheme 2, wherein Re is H or methyl, R³ is NR^{3a}R^{3b}, R⁴ is C₁₋₃ alkyl, bromo, chloro, fluoro, cyano, OMe, or CF₃, and all other variables are as defined hereinabove. In other embodiments, there is provided a compound of formula IA or IB, wherein X¹ is N, X² is CH, Ar is IIa, R¹ is selected from Scheme 1, R² is (alkylene)₁₋₂-heterocyclyl, and heterocyclyl is selected from Scheme 2, wherein Re is H or methyl, R³ is NR^{3a}R^{3b}, R⁴ is C₁₋₃ alkyl, bromo, chloro, fluoro, cyano, OMe, or CF₃, and all other variables are as defined hereinabove.

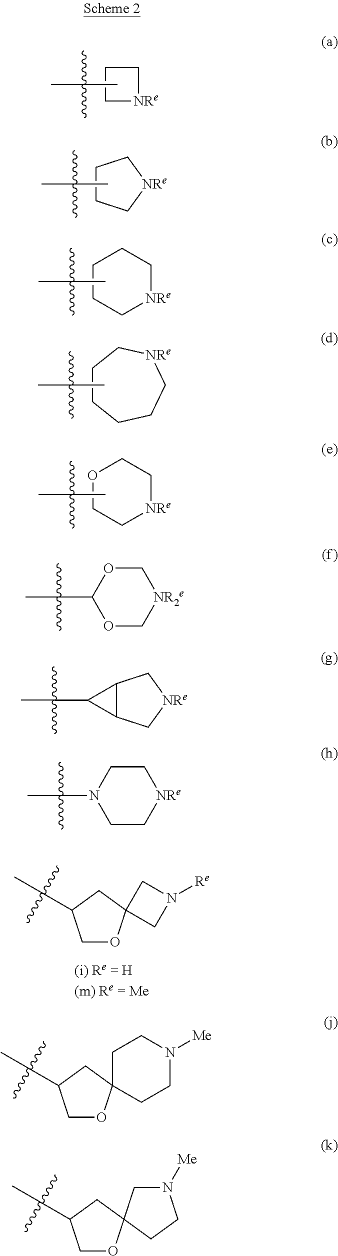

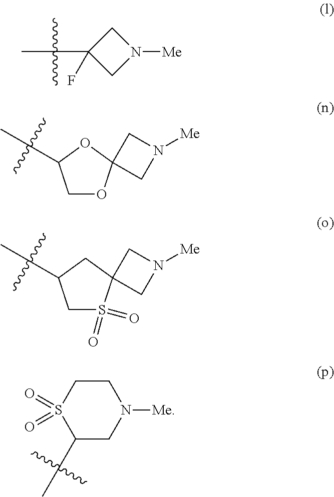

In another embodiment of the present invention there is provided a compound of formula IA or IB, wherein X¹ is N, X² is CH, Ar is IIa, R¹ is C₁₋₃ alkyl, R² is selected from the group consisting of 1-morpholin-2-ylethyl, 1-morpholin-2-ylmethyl, (5-amino-1,3-dioxan-2-yl)ethyl, (5-amino-1,3-dioxan-2-yl)ethyl, 5,5-difluoro-1-methylpiperidin-3-yl, and 4,4-difluoro-1-methylpyrrolidin-3-yl, R³ is NR^{3a}R^{3b}, R⁴ is C₁₋₃ alkyl, bromo, chloro, fluoro, cyano, OMe, or CF₃, and all other variables are as defined hereinabove. In other embodiments, there is provided a compound of formula IA or IB, wherein X¹ is N, X² is CH, Ar is IIa, R¹ is selected from Scheme 1, R² is selected from the group consisting of 1-morpholin-2-ylethyl, 1-morpholin-2-ylmethyl, (5-amino-1,3-dioxan-2-yl)ethyl, (5-amino-1,3-dioxan-2-yl)ethyl, 5,5-difluoro-1-methylpiperidin-3-yl, and 4,4-difluoro-1-methylpyrrolidin-3-yl, R³ is NR^{3a}R^{3b}, R⁴ is C₁₋₃ alkyl, bromo, chloro, fluoro, cyano, OMe, or CF₃, and all other variables are as defined hereinabove.

In another embodiment there is a compound of formula IA or IB, wherein X¹ is N, X² is CH, Ar is IIa, R³ is NR^{3a}R^{3b}, R⁴ is C₁₋₃ alkyl, bromo, chloro, fluoro, cyano, OMe, or CF₃, R¹ is (alkylene)₀₋₆R^{1a}, wherein R^{1a} is hydrogen (i.e., in this embodiment R¹ is hydrogen or C₁₋₆ alkyl), and R² is selected from the group consisting of (iv) (alkylene)₂₋₃NR^{a}R^{b}, wherein the alkylene chain is optionally substituted by a hydroxyl, (v) (alkylene)₂₋₃OR⁵, wherein R⁵ is (alkylene)₂₋₄NR^{a}R^{b} or a heterocycle selected from azetidine, pyrrolidine, piperidine, or azepane; (vi) (alkylene)₀₋₃-(C₄₋₆-cycloalkyl NR^{a}R^{b}), and (vii) (alkylene)₀₋₃-heterocyclyl, wherein heterocyclyl refers to azetidinyl, pyrrolidinyl, piperidinyl, azepanyl, morpholinyl, 1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl, 5,8-dioxa-2-azaspiro[3.4]octan-6-yl, 5,5-dioxido-5-thia-2-azaspiro[3.4]octan-7-yl, thiomorpholinyl, or piperazinyl, each optionally substituted by one or more moieties selected from the group consisting of C₁₋₃ alkyl, C(═O)CHR^{f}NH₂ (wherein R^{f} is hydrogen or C₁₋₃ alkyl), halogen, oxo, hydroxyl, amino, C₁₋₃ alkylamino, C₁₋₃ dialkylamino, and C₁₋₆-hydroxyalkyl. In one subembodiment, R¹ is hydrogen or C₁₋₆ alkyl and R² is (alkylene)₁₋₂-heterocyclyl, wherein heterocyclyl refers to 5-amino-1,3-dioxolan-2-yl. In another subembodiment, R¹ is hydrogen or C₁₋₆ alkyl and R² is (alkylene)₁₋₂-heterocyclyl, wherein heterocyclyl refers to morpholinyl. In another subembodiment, R¹ is hydrogen or C₁₋₆ alkyl and R² is 1-morpholin-2-ylmethyl. In another subembodiment, R¹ is hydrogen or C₁₋₆ alkyl and R² is 1-morpholin-2-ylethyl. In another subembodiment, R¹ is hydrogen or C₁₋₆ alkyl and R² is 4,4-difluoro-1-methylpyrrolidin-3-yl. In another subembodiment, R¹ is hydrogen or C₁₋₆ alkyl and R² is 5,5-difluoro-1-methylpiperidin-3-yl. In another subembodiment, R¹ is hydrogen or C₁₋₆ alkyl and R² is (5-amino-1,3-dioxan-2-yl)methyl. In another subembodiment, R¹ is hydrogen or C₁₋₆ alkyl and R² is (5-amino-1,3-dioxan-2-yl)ethyl.

In another embodiment of the present invention there is provided a compound of formula IA or IB wherein X¹ is N, X² is CH, Ar is IIb, R³ is NR^{3a}R^{3b}, R⁴ is C₁₋₃ alkyl, bromo, chloro, fluoro, cyano, OMe, or CF₃, R¹ is selected from Scheme 1, and all other variables are as defined hereinabove.

In another embodiment of the present invention there is provided a compound of formula IA or IB wherein X¹ and X² are N, Ar is IIc, R³ is NR^{3a}R^{3b}, R⁴ is C₁₋₃ alkyl, bromo, chloro, fluoro, cyano, OMe or CF₃, R¹ is selected from Scheme 1, and all other variables are as defined hereinabove.

In another embodiment of the present invention there is provided a compound of formula IA or IB, wherein X¹ is N; X² is CH; Ar is IIb; R⁴ is methyl or Cl; R¹ is selected from Scheme 1; and R^{3a} and R^{3b} are independently each hydrogen or optionally substituted C₁₋₆ alkyl, or R^{3a} and R^{3b} together with the nitrogen to which they are attached form pyrrolidine, piperidine, morpholine, piperazine, N-methyl-piperazine, or 1,1-dioxo-thiomorpholine.

In another embodiment of the present invention there is provided a compound of formula IA or IB wherein X¹ is N; X² is CH; Ar is IIc; R⁴ is methyl or Cl; R¹ is selected from Scheme 1; and R^{3a} and R^{3b} are independently hydrogen or optionally substituted C₁₋₆ alkyl, or R^{3a} and R^{3b} together with the nitrogen to which they are attached form pyrrolidine, piperidine, morpholine, piperazine, N-methyl-piperazine, or 1,1-dioxo-thiomorpholine.

In another embodiment of the present invention there is provided a compound of formula IA or IB wherein X¹ is N; X² is CH; Ar is IId; R⁴ is methyl or chloro; R¹ is selected from Scheme 1; and R^{3a} and R^{3b} are independently hydrogen or optionally substituted C₁₋₆ alkyl, or R^{3a} and R^{3b} together with the nitrogen to which they are attached form pyrrolidine, piperidine, morpholine, piperazine, N-methyl-piperazine, or 1,1-dioxo-thiomorpholine.

In some embodiments, R¹ is C₁₋₃ alkyl. In other embodiments, R¹ is (alkylene)₀₋₆R^{3a}, wherein R^{1a} is heterocycle selected from the group consisting of azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, N—C₁₋₆ alkyl piperazinyl, tetrahydropyranyl, tetrahydrofuranyl, 3-azabicyclo[3.1.0]hexan-6-yl, and 1,3-dioxolan-2-yl, each said heterocycle optionally substituted by oxo, hydroxyl, amino, C₁₋₃ alkylamino, or C₁₋₃ dialkylamino. In still other embodiments, R¹ is (alkylene)₂₋₄R^{1a}.

In some embodiments, R² is H, C₁₋₆ alkyl, or C₃₋₇ cycloalkyl. In other embodiments, R² is selected from the group consisting of 1-morpholin-2-ylethyl, 1-morpholin-2-ylmethyl, (5-amino-1,3-dioxan-2-yl)ethyl, and (5-amino-1,3-dioxan-2-yl)ethyl.

In some embodiments, R^{3a} and R^{3b} are independently hydrogen, C₁₋₄ alkyl (optionally substituted with hydroxyl), C₁₋₄ haloalkyl, or C₃₋₆ cycloalkyl, or R^{3a} and R^{3b} taken together with the nitrogen to which they are attached form a cyclic amine optionally substituted with C₁₋₄ alkyl or two oxo groups.

In some embodiments, there is provided a compound of formula II:

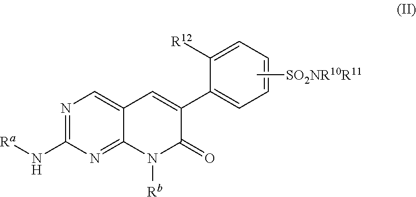

wherein
R^{a} is C₂₋₄ alkyl, (alkylene)₀₋₂-HetA, wherein HetA is azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, N—C₁₋₆ alkyl piperazinyl, tetrahydropyranyl, tetrahydrofuranyl, 3-azabicyclo[3.1.0]hexan-6-yl, and 5-amino-1,3-dioxolan-2-yl, each said heterocycle optionally substituted by oxo, hydroxyl, amino, C₁₋₃ alkylamino, or C₁₋₃ dialkylamino;
R^{b} is C₁₋₄ alkyl or (alkylene)₁₋₂-HetB, wherein HetB is azetidinyl, pyrrolidinyl, piperidinyl, azepanyl, morpholinyl, 1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl, 5,8-dioxa-2-azaspiro[3.4]octan-6-yl, 5,5-dioxido-5-thia-2-azaspiro[3.4]octan-7-yl, thiomorpholinyl, or piperazinyl, each optionally substituted by one or more moieties selected from the group consisting of C₁₋₆ alkyl, halogen, oxo, hydroxyl, amino, C₁₋₃ alkylamino, C₁₋₃ dialkylamino, and C₁₋₆-hydroxyalkyl;
R¹⁰ and R¹¹ are independently hydrogen, C₁₋₄ alkyl, or C₃₋₆ cycloalkyl; or R¹⁰ and R¹¹ taken together with the nitrogen to which they are attached form a 4- to 7-membered heterocyclyl optionally containing on oxygen, sulfur, or additional nitrogen ring member, wherein the ring is optionally substituted with C₁₋₄ alkyl or one or two oxo groups;
R¹² is hydrogen, C₁₋₄ alkyl, or halo;
   or a pharmaceutically acceptable salt thereof.

In some embodiments of formula II, R^{a} is ethyl, propyl, or isopropyl; -ethylene-HetA; or HetA. In other embodiments, R^{a} is ethyl, (N-methylpiperidin-4-yl)ethyl, (N-ethylpiperidin-4-yl)ethyl, or 3-aza-bicyclo[3.1.0]hexan-6-yl. In other embodiments, R^{a} is ethyl or (N-methylpiperidin-4-yl)ethyl. In some embodiments, R^{b} is methyl, ethyl, or isopropyl, or (alkylene)₁₋₂-HetB. In some embodiments, HetB is piperidinyl, 1,3-dioxolan-2-yl, or morpholinyl, each optionally substituted with one or two groups selected from amino and halo. In some embodiments, R^{b} is methyl or ethyl. In some embodiments, R¹⁰ and R¹¹ are independently hydrogen, methyl, ethyl, isopropyl, tert-butyl, cyclopropyl, cyclobutyl, cyclopentyl, or cyclohexyl, or R¹⁰ and R¹¹ taken together with the nitrogen to which they are attached form pyrrolidinyl or morpholinyl. In some embodiments, R¹⁰ is hydrogen or methyl, R¹¹ is methyl, ethyl, isopropyl, or cyclopropyl, or R¹⁰ and R¹¹ taken together with the nitrogen to which they are attached form pyrrolidinyl or morpholinyl. In some embodiments, R¹² is hydrogen, methyl, or halo. In other embodiments, R¹² is methyl or chloro. In some embodiments, the —SO₂NR¹⁰R¹¹ group is meta to R¹². In other embodiments, the —SO₂NR¹⁰R¹¹ group is para to R¹².

In another embodiment of the present invention there is provided a compound selected from I-1 to I-6 of TABLE I, or a pharmaceutically acceptable salt thereof. In another embodiment of the present invention there is provided a compound selected from I-1 to I-14 of TABLE I, or a pharmaceutically acceptable salt thereof. In other embodiments, there is provided a compound of Table IA, or a pharmaceutically acceptable salt thereof.

In any method, formulation, kit or other embodiment provided herein which references to formula I, IA or IB, it is understood that the same method, formulation, kit or embodiment is in one aspect equally applicable with reference to any formula or compound detailed herein, such as formula II or a compound of TABLE I or IA, or a pharmaceutically acceptable salt thereof.

In another embodiment there is provided a method of inhibiting PAK1 activity in a cell comprising treating the cell with an inhibitory amount of a compound of (a) formula IA or (b) formula IB, or a pharmaceutically acceptable salt thereof, wherein each variable is as defined hereinabove.

In another embodiment of the present invention there is provided a method of inhibiting PAK activity in a patient in need thereof comprising the step of administering to said patient an effective amount of a compound of (a) formula IA or (b) formula IB, or a pharmaceutically acceptable salt thereof, wherein each variable is as defined hereinabove.

In another embodiment of the preset invention there is provided a method of treating or ameliorating the severity of cancer or a hyperproliferative disorder in a patient in need thereof comprising administering to said patient an effective amount of a compound of (a) formula IA or (b) formula IB, or a pharmaceutically acceptable salt thereof, wherein each variable is as defined hereinabove.

In another embodiment of the present invention there is provided a method of treating or ameliorating the severity of cancer or a hyperproliferative disorder selected from the group consisting of adenoma, bladder cancer, brain cancer, breast cancer, colon cancer, epidermal carcinoma, follicular carcinoma, cancer of the genitourinary tract, glioblastoma, Hodgkin's disease, head and neck cancers, heptoma, keratoacanthoma, kidney cancer, large cell carcinoma, leukemias, lung adenocarcinoma, lung cancer, lymphoid disorders, melanoma and non-melanoma skin cancer, myelodysplastic syndrome, neuroblastoma, non-Hodgkins lymphoma, ovarian cancer, papillary carcinoma, pancreatic cancer, prostate cancer, rectal cancer, sarcoma, small cell carcinoma, testicular cancer, tetracarcinomas, thyroid cancer, and undifferentiated carcinoma in a patient in need thereof comprising administering to said patient an effective amount of a compound of (a) formula IA or (b) formula IB, or a pharmaceutically acceptable salt thereof, wherein each variable is as defined hereinabove.

In another embodiment of the present invention there is provided a method of treating or ameliorating the severity of cancer or a hyperproliferative disorder selected from the group consisting of lung cancer, breast cancer, ovarian cancer, bladder cancer and head and neck cancer in a patient in need thereof comprising administering to said patient an effective amount of a compound of (a) formula IA or (b) formula IB, or a pharmaceutically acceptable salt thereof, wherein each variable is as defined hereinabove.

In another embodiment of the present invention there is provided a method of treating or ameliorating the severity of cancer or a hyperproliferative disorder selected from the group consisting of primary breast adenocarcinoma, squamous non-small cell lung cancer or a squamous head and neck cancer in a patient in need thereof comprising administering to said patient an effective amount of a compound of (a) formula IA or (b) formula IB, or a pharmaceutically acceptable salt thereof, wherein each variable is as defined hereinabove.

In another embodiment of the present invention there is provided a method of treating or ameliorating the severity of cancer or a hyperproliferative disorder in a patient in need thereof comprising co-administering to said patient an effective amount of a compound of (a) formula IA or (b) formula IB, or a pharmaceutically acceptable salt thereof, wherein each variable is as defined hereinabove, with at least one other chemotherapeutic agent.

In another embodiment of the present invention there is provided a method of treating or ameliorating the severity of cancer or a hyperproliferative disorder in a patient in need thereof comprising co-administering to said patient an effective amount of a compound of (a) formula IA or (b) formula IB, or a pharmaceutically acceptable salt thereof, wherein each variable is as defined hereinabove with at least one other chemotherapeutic agent selected from the group consisting of inhibitor of apoptosis proteins (TAP), an EGFR inhibitor or antagonist, an inhibitor of Ras/Raf/Mek/Erk signaling cascade, an inhibitor of Akt kinase and a Src kinase inhibitor.

In any of the methods detailed herein with reference to a patient, in one embodiment the patient is a human patient.

In another embodiment of the present invention there is provided a pharmaceutical formulation containing a compound of (a) formula IA or (b) formula IB, or a pharmaceutically acceptable salt thereof, wherein each variable is as defined hereinabove and at least one pharmaceutically acceptable carrier, excipient or diluent.

The term “alkyl” as used herein alone or in combination with other groups, denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₆ alkyl” as used herein refers to an alkyl composed of 1 to 6 carbons. Examples of alkyl groups include, but are not limited to, methyl, ethyl, propyl, i-propyl, n-butyl, i-butyl, t-butyl, neopentyl, hexyl, and octyl.

The term “haloalkyl” as used herein denotes an alkyl group as defined above wherein at least one hydrogen atom is substituted by a halogen. Examples are 1-fluoromethyl, 1-chloromethyl, 1-bromomethyl, 1-iodomethyl, difluoromethyl, trifluoromethyl, trichloromethyl, 1-fluoroethyl, 1-chloroethyl, 2-fluoroethyl, 2-chloroethyl, 2-bromoethyl, 2,2-dichloroethyl, 3-bromopropyl or 2,2,2-trifluoroethyl.

The term “cycloalkyl” denotes a monovalent, saturated, monocyclic or bicyclic hydrocarbon group of 3 to 10 ring carbon atoms. Polycyclic cycloalkyl groups include spirocyclic, fused bicyclic, or fused polycyclic systems consisting of two saturated carbocycles having one two or more carbon atoms in common. Particular cycloalkyl groups are monocyclic. “C₃₋₇ cycloalkyl” as used herein refers to a cycloalkyl composed of 3 to 7 carbons in the carbocyclic ring. Examples for monocyclic cycloalkyl are cyclopropyl, cyclobutanyl, cyclopentyl, cyclohexyl or cycloheptyl. Examples for bicyclic cycloalkyl are bicyclo[2.2.1]heptanyl or bicyclo[2.2.2]octanyl.

The term “alkoxy” as used herein means an —O-alkyl group which is attached to the remainder of the molecule by an oxygen atom, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “cyclic amine” denotes a saturated carbon ring, containing from 3 to 6 carbon atoms as defined above, and wherein at least one of the carbon atoms is replaced by a nitrogen atom and one or more other carbon atoms are optionally replaced by a heteroatom selected from the group consisting of N, O or S(O)₀₋₂, for example, piperidine, piperazine, morpholine, thiomorpholine, di-oxo-thiomorpholine, pyrrolidine, pyrazoline, imidazolidine, or azetidine, wherein the cyclic carbon atoms are optionally substituted by one or more substituents, selected from the group consisting of halogen, hydroxy, phenyl, lower alkyl, and lower alkoxy, or two hydrogen atoms on a carbon are both replaced by oxo (═O). When the cyclic amine is a piperazine, one nitrogen atom can be optionally substituted by C₁₋₆ alkyl, C₁₋₆ acyl, C₁₋₆ alkylsulfonyl. The term “cyclic amine” also denotes a four to seven-membered ring containing a nitrogen atom and optionally a second heteroatom selected from O, NR^{x} (where R^{x} is, for example, hydrogen, C₁₋₆ alkyl, —C(O)₁₋₂C₁₋₆ alkyl, or —SO₁₋₂C₁₋₆ alkyl) or S(O)₀₋₂, and, unless specifically limited, optionally substituted either by one or more substituents, selected from the group consisting of halogen, hydroxy, and NR^{y}R^{z} (wherein R^{y} and R^{z} are independently hydrogen or C₁₋₃ alkyl), phenyl, lower alkyl, and lower alkoxy, or two hydrogen atoms on a carbon are both replaced by oxo (═O). When the cyclic amine is a piperazine, one nitrogen atom can be optionally substituted by C₁₋₆ alkyl, C₁₋₆ acyl, or C₁₋₆ alkylsulfonyl.

The term “oxo” as used herein refers to a doubly bonded oxygen such as “C═O” (i.e., a carbonyl group when the oxo is attached to a carbon) wherein it is understood that this is equivalent to two hydroxyl groups attached to the same carbon are equivalent.

The term “halogen” or “halo” as used herein means fluorine, chlorine, bromine, or iodine. The term “halo,” “halogen,” and “halide” are used interchangeably herein and denote fluoro, chloro, bromo, or iodo.

“Heterocycle” or “heterocyclic ring” or “heterocyclyl” as used herein means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. When the heterocycle is bicyclic one ring can lack a heteroatom and be aromatic, partially unsaturated, or saturated but heterocycle is attached to the remainder of the molecule at the heterocyclic ring.

The terms “amino,” “alkylamino,” and “dialkylamino” as used herein refer to —NH₂, —NHR, and —NR₂ respectively, wherein R is alkyl as defined above. The two alkyl groups attached to a nitrogen in a dialkyl moiety can be the same or different. The terms “aminoalkyl,” “alkylaminoalkyl,” and “dialkylaminoalkyl” as used herein refer to NH₂(CH₂)ₙ—, RHN(CH₂)ₙ—, and R₂N(CH₂)ₙ—, respectively, wherein n is 1 to 6 and R is alkyl as defined above. “C₁₋₁₀ alkylamino” as used herein refers to an alkylamino moiety wherein alkyl is C₁₋₁₀. The term “phenylamino” as used herein refers to —NHPh wherein Ph represents an optionally substituted phenyl group.

The term “alkylene” as used herein denotes a divalent saturated linear hydrocarbon radical of 1 to 10 carbon atoms (e.g., (CH₂), where r is 1-10) or a branched saturated divalent hydrocarbon radical of 2 to 10 carbon atoms (e.g., —CHMeCH₂CH— or —CH₂CH(i-Pr)CH₂—), unless otherwise indicated. C₀₋₄ alkylene or (alkylene)₀₋₄ refers to a linear or branched saturated divalent hydrocarbon radical comprising 1-4 carbon atoms or, in the case of Cₒ, the alkylene radical is omitted. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, and 2-ethylbutylene.

The term “(C₄₋₆-cycloalkyl-NR^{a}R^{b})” as used herein refers to cycloalkyl as defined herein substituted by NR^{a}R^{b}. An exemplary structure is depicted as (i) in Scheme 3 below. The term “(alkylene)_{x-y}-(C₄₋₆-cycloalkyl-NR^{a}R^{b})” as used herein refers to (C₄₋₆-cycloalkyl-NR^{a}R^{b}) as defined above, linked to an optionally substituted alkylene radical “(alkylene)_{x-y}” as defined herein with the understanding that the attachment point of the aminocycloalkylalkyl moiety will be on the alkylene radical. An exemplary structure is shown as (ii) in Scheme 3.

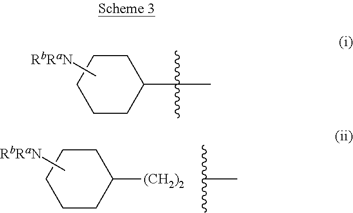

The term “(alkylene)_{x-y}-heterocyclyl,” where x and y are integers greater than or equal to zero and y>x, denotes the radical of the formula R′—R″—, wherein R′ is an optionally substituted heterocyclic radical as defined herein, and R″ is an alkylene radical as defined herein and the attachment point of the heterocycyl radical will be on the alkylene radical. In some embodiments, the term “heterocyclyl” in this context refers to a heterocycle selected from the group consisting of azetidinyl (a), pyrrolidinyl (b), piperidinyl (c), azepanyl (d), morpholinyl (e), 5-amino-1,3-dioxolan-2-yl (f), 3-aza-bicyclo[3.1.0]hexan-6-yl (g), piperazinyl (h), 5-oxa-2-azaspiro[3.4]octan-7-yl (i), 1-oxa-8-azaspiro[4.5]decan-3-yl (j), 1-oxa-7-azaspiro[4.4]nonan-3-yl (k), 3-fluoro-1-methylazetidin-3-yl (l), 2-methyl-5-oxa-2-azaspiro[3.4]octan-7-yl (m), 2-methyl-5,8-dioxa-2-azaspiro[3.4]octan-6-yl (n), 2-methyl-5,5-dioxido-5-thia-2-azaspiro[3.4]octan-7-yl (o), and 4-methyl-1,1-dioxidothiomorpholin-2-yl (p), as shown in Scheme 2 above. In some embodiments, optionally substituted heterocyclyl refers at least to substitution by optionally substituted by hydroxyl, halogen, amino, C₁₋₃ alkylamino, C₁₋₃ dialkylamino, C₁₋₆ alkyl, or C₁₋₆-hydroxyalkyl, or two hydrogen atoms on a carbon are replaced by an oxo moiety. In other embodiments, R^{e} as in Scheme 2 is hydrogen, C₁₋₃ alkyl, or C₁₋₃ alkylsulfonyl.

The terms “treat” and “treatment” refer to therapeutic treatment wherein the object is to slow down (lessen) an undesired physiological change or disorder, such as the spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “treating” or “treatment” of a disease state includes (1) inhibiting the disease state, i.e., arresting the development of the disease state or its clinical symptoms, or (2) relieving the disease state, i.e., causing temporary or permanent regression of the disease state or its clinical symptoms.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include erlotinib (TARCEVA®, Genentech/OSI Pharm.), bortezomib (VELCADE®, Millennium Pharm.), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ11 and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; (ix) anti-angiogenic agents such as bevacizumab (AVASTIN®), Genentech); and (x) pharmaceutically acceptable salts, acids and derivatives of any of the above.

### Compounds and Preparation

Examples of representative compounds within the scope of the invention are provided in the following tables. These examples and preparations which follow are provided to enable those skilled in the art to more clearly understand and to practice the present invention. They should not be considered as limiting the scope of the invention, but merely as being illustrative and representative thereof.

TABLE I

| | | PAK1¹ | |
|---|---|---|---|
| Cpd. | | IC₅₀ | |
| No. | Structure | (μM) | Name |
| I-1. | 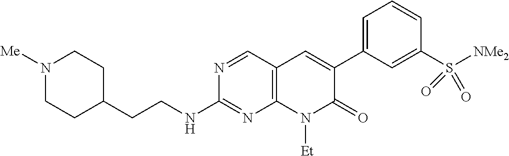 [SMILES: CCn1c(=O)c(-c2cccc(S(=O)(=O)N(C)C)c2)cc2cnc(NCCC3CCN(C)CC3)nc21] | 0.182 | 3-[8-ethyl-2-[2-(1-methyl-4- piperidyl)ethylamino]-7-oxo- pyrido[2,3-d]pyrimidin-6-yl]- N,N-dimethyl- benzenesulfonamide |
| I-2. | 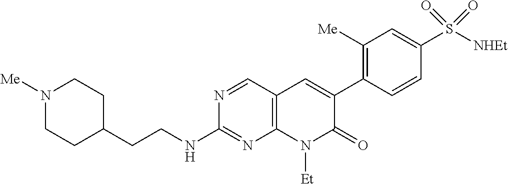 | 0.133 | N-ethyl-4-[8-ethyl-2-[2-(1- methyl-4-piperidyl)ethylamino]- 7-oxo-pyrido[2,3-d]pyrimidin-6- yl]-3-methyl- benzenesulfonamide |
| I-3. | 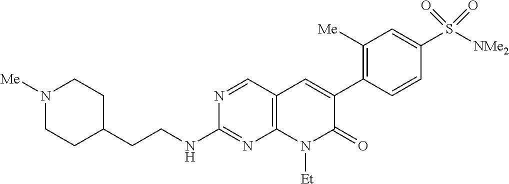 [SMILES: CCn1c(=O)c(-c2ccc(S(=O)(=O)N(C)C)cc2C)cc2cnc(NCCC3CCN(C)CC3)nc21] | 1.32 | 4-[8-ethyl-2-[2-(1-methyl-4- piperidyl)ethylamino]-7-oxo- pyrido[2,3-d]pyrimidin-6-yl]- N,N,3-trimethyl- benzenesulfonamide |
| I-4. | 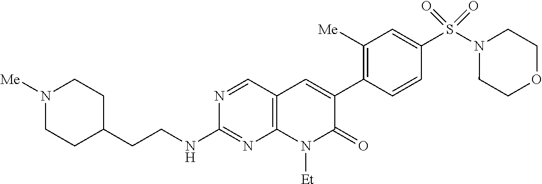 [SMILES: CCn1c(=O)c(-c2ccc(S(=O)(=O)N3CCOCC3)cc2C)cc2cnc(NCCC3CCN(C)CC3)nc21] | 0.751 | 8-ethyl-6-(2-methyl-4- morpholinosulfonyl-phenyl)-2- [2-(1-methyl-4- piperidyl)ethylamino]pyrido[2,3- d]pyrimidin-7-one |
| I-5. | 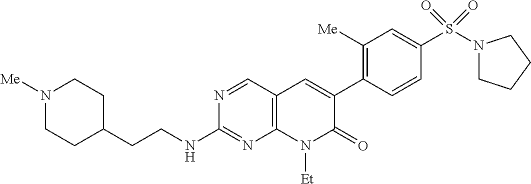 [SMILES: CCn1c(=O)c(-c2ccc(S(=O)(=O)N3CCCC3)cc2C)cc2cnc(NCCC3CCN(C)CC3)nc21] | 0.460 | 8-ethyl-2-[2-(1-methyl-4- piperidyl)ethylamino]-6-(2- methyl-4-pyrrolidin-1- ylsulfonyl-phenyl)pyrido[2,3- d]pyrimidin-7-one |
| I-6. | 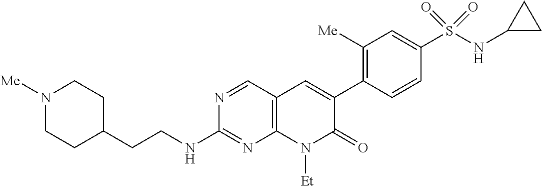 [SMILES: CCn1c(=O)c(-c2ccc(S(=O)(=O)NC3CC3)cc2C)cc2cnc(NCCC3CCN(C)CC3)nc21] | 0.0771 | N-cyclopropyl-4-[8-ethyl-2-[2- (1-methyl-4- piperidyl)ethylamino]-7-oxo- pyrido[2,3-d]pyrimidin-6-yl]-3- methyl-benzenesulfonamide |
| I-7. | 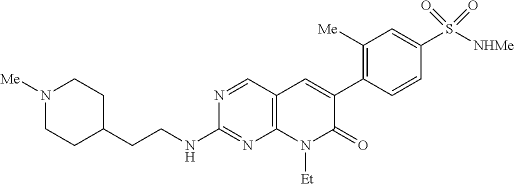 [SMILES: CCn1c(=O)c(-c2ccc(S(=O)(=O)NC)cc2C)cc2cnc(NCCC3CCN(C)CC3)nc21] | 0.582 | 4-[8-ethyl-2-[2-(1-methyl-4- piperidyl)ethylamino]-7-oxo- pyrido[2,3-d]pyrimidin-6-yl]- N,3-dimethyl- benzenesulfonamide |
| I-8. | 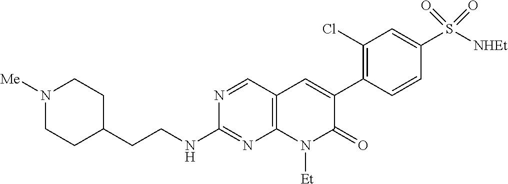 | 0.0223 | 3-chloro-N-ethyl-4-[8-ethyl-2- [2-(1-methyl-4- piperidyl)ethylamino]-7-oxo- pyrido[2,3-d]pyrimidin-6- yl]benzenesulfonamide |
| I-9. | 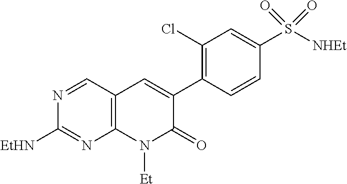 | 0.131 | 3-chloro-N-ethyl-4-[8-ethyl-2- (ethylamino)-7-oxo-pyrido[2,3- d]pyrimidin-6- yl]benzenesulfonamide |
| I-10. | 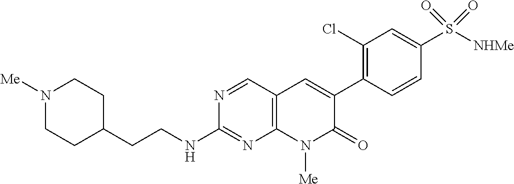 [SMILES: CNS(=O)(=O)c1ccc(-c2cc3cnc(NCCC4CCN(C)CC4)nc3n(C)c2=O)c(Cl)c1] | 0.82 | 3-chloro-N-methyl-4-[8-methyl- 2-[2-(1-methyl-4- piperidyl)ethylamino]-7-oxo- pyrido[2,3-d]pyrimidin-6- yl]benzenesulfonamide |
| I-11. | 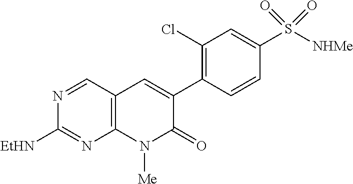 | 1.60 | 3-chloro-4-[2-(ethylamino)-8- methyl-7-oxo-pyrido[2,3- d]pyrimidin-6-yl]-N-methyl- benzenesulfonamide |
| I-12. | 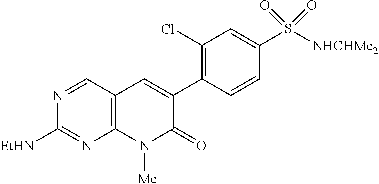 | 0.177 | 3-chloro-4-[2-(ethylamino)-8- methyl-7-oxo-pyrido[2,3- d]pyrimidin-6-yl]-N-isopropyl- benzenesulfonamide |
| I-13. | 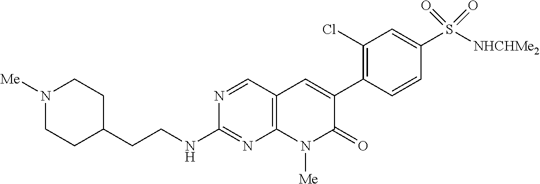 | 0.0804 | 3-chloro-N-isopropyl-4-[8- methyl-2-[2-(1-methyl-4- piperidyl)ethylamino]-7-oxo- pyrido[2,3-d]pyrimidin-6- yl]benzenesulfonamide |
| I-14. | 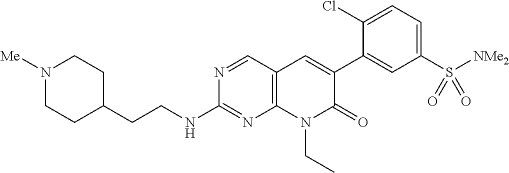 [SMILES: CCn1c(=O)c(-c2cc(S(=O)(=O)N(C)C)ccc2Cl)cc2cnc(NCCC3CCN(C)CC3)nc21] | 0.31 | 4-chloro-3-[8-ethyl-2-[2-(1- methyl-4-piperidyl)ethylamino]- 7-oxo-pyrido[2,3-d]pyrimidin-6- yl]-N,N-dimethyl- benzenesulfonamide |

¹Biological Example 1 - PAK1-KD (kinase domain) IC₅₀ Zlyte assay Protocol

Other examples of compounds within the scope of the invention include those in Table IA:

TABLE IA

| | |
|---|---|
| 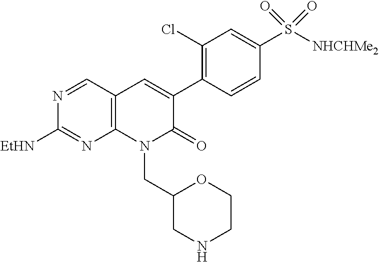 | 3-Chloro-4-(2-ethylamino-8-morpholin-2- ylmethyl-7-oxo-7,8-dihydro-pyrido[2,3- d]pyrimidin-6-yl)-N-isopropyl- benzenesulfonamide |
| 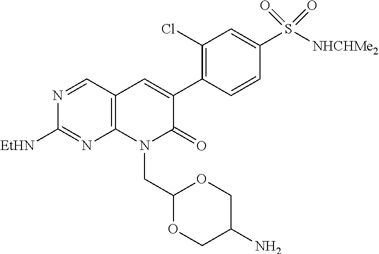 | 4-[8-(5-Amino-[1,3]dioxan-2-ylmethyl)-2- ethylamino-7-oxo-7,8-dihydro-pyrido[2,3- d]pyrimidin-6-yl]-3-chloro-N-isopropyl- benzenesulfonamide |
| 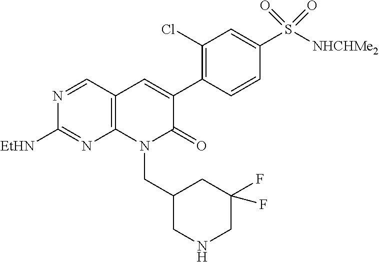 | 3-Chloro-4-[8-(4,4-difluoro-piperidin-3- ylmethyl)-2-ethylamino-7-oxo-7,8-dihydro- pyrido[2,3-d]pyrimidin-6-yl]-N-isopropyl- benzenesulfonamide |

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. Larock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, about 20° C.

Some compounds in following schemes are depicted with generalized substituents; however, one skilled in the art will immediately appreciate that the nature of the R groups can varied to afford the various compounds contemplated in this invention.

The reaction conditions are exemplary and alternative conditions are well known. The reaction sequences in the following examples are not meant to limit the scope of the invention as set forth in the claims.

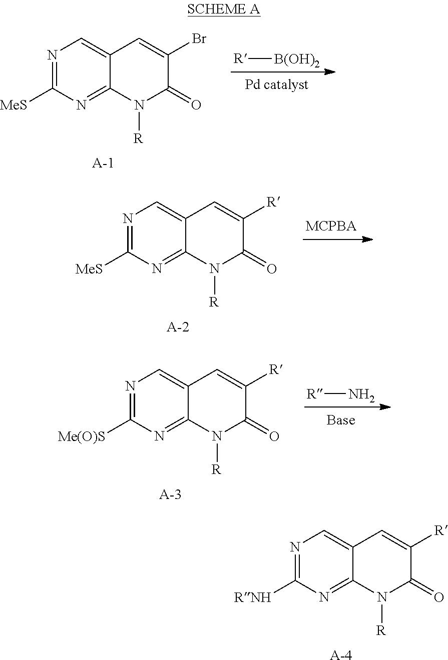

Generally, compounds of formula I as described herein can be prepared from 6-bromo-8-ethyl-2-(methylthio)pyrido[2,3-d]pyrimidin-7(8H)-one (CASRN 851756-48-4) or 6-bromo-8-methyl-2-(methylthio)pyrido[2,3-d]pyrimidin-7(8H)-one (CASRN 1232030-55-5), which are prepared by contacting 6-bromo-2-(methylthio)pyrido[2,3-d]pyrimidin-7(8H)-one (CASRN 352328-87-1) with a base capable of deprotonating the lactam, and reacting the resulting salt with an alkylating agent. One skilled in the art will appreciate this provides a flexible process for a variety of substituents on the lactam nitrogen and the current process is not limited to methyl or ethyl. Introduction of an aryl or heteroaryl sulfone at the 6-position is conveniently accomplished by palladium-catalyzed Suzuki couplings of A-1 and a (hetero)aryl boronic acid substituted by a sulfone group to afford A-2. These boronic acids are available from a variety of commercial sources. Oxidation of the methylthio substituent to a sulfoxide is readily accomplished with an oxidant such as m-chloroperoxybenzoic acid, which allows for introduction of a substituted amine at the 2-position by a direct displacement. One skilled in the art will appreciate that the sequence of the three steps can be varied without departing from the general process depicted in SCHEME A.

The Suzuki coupling reaction is conveniently carried out in a solvent such as toluene, dioxane, dimethoxyethane, or THF, using a suitable catalyst, for example bis-(tri-o-tolylphosphine)-palladium-(II)-chloride, tris-(dibenzylideneacetone)-dipalladium(0)/tris-μ-tolylphosphine, tris-(dibenzylideneacetone)-dipalladium(0)/tris-(2-furyl)phosphine, tris-(dibenzylideneacetone)-dipalladium(1)/2,2′-bis-(diphenylphosphino)-1,1′-binaphthyl, tetrakis-(triphenylphosphine)-palladium(0), 1,1′-bis-(diphenylphosphino)-ferrocene-palladium-dichloride, or palladium-II-acetate/1,3-bis-(triphenylphosphino)-propane, preferably in the presence of a base such as sodium tert-butoxide, bis-(trimethylsilyl)-lithium amide, potassium carbonate, cesium carbonate, or triethylamine, at a temperature between 0 and 150° C., preferably 20 to 100° C.

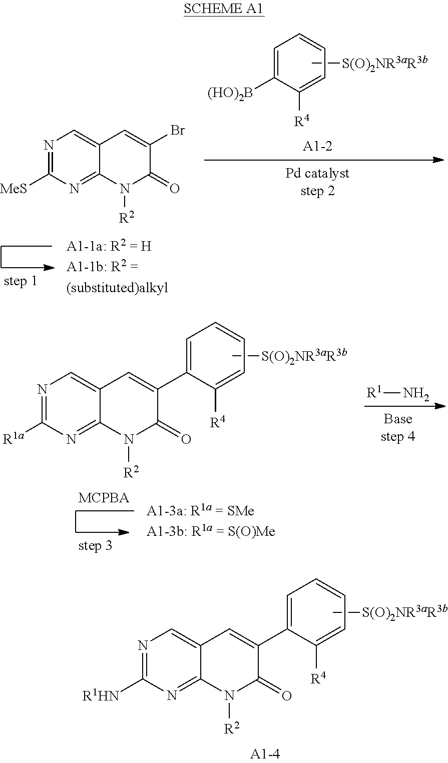

Compounds of formula I as described herein may also be prepared from 6-bromo-2-(methylthio)pyrido[2,3-d]pyrimidin-7(8H)-one (A1-1a CASRN352328-87-1) as shown in Scheme A1. Alkylation of the lactam nitrogen provides a general method to incorporate the requisite substitution at this position. Amide alkylations are commonly carried out in aprotic solvents such as THF, DMF, DMSO, NMP, and mixtures thereof, at temperatures between −78° C. and 100° C. Typical bases include Cs₂CO₃, sodium hydride, potassium hydride, sodium methoxide, potassium tert-butoxide, lithium hexamethyldisilazide, sodium hexamethyldisilazide, and potassium hexamethyldisilazide. The Mitsunobu coupling protocol also can be utilized. Thus, 6-bromo-8-ethyl-2-(methylthio)pyrido[2,3-d]pyrimidin-7(8H)-one (A1-1b, R²=Et, CASRN 851756-48-4) or 6-bromo-8-methyl-2-(methylthio)pyrido[2,3-d]pyrimidin-7(8H)-one (A1-1b, R²=Me, CASRN 1232030-55-5) are obtained by alkylation with ethyl iodide or methyl iodide, respectively.

Introduction of an aryl or heteroaryl sulfonamide at the 6-position is conveniently accomplished by palladium-catalyzed Suzuki-Miyaura couplings of A1-1b and an aryl boronic acid A1-2, or a corresponding substituted heteroarylboronic acid, wherein one or two carbon atoms of A1-2 are replaced by a nitrogen, to afford pyridinyl, pyridine-N-oxide, pyridinone, pyrimidinyl, pyridazinyl, or pyrazinyl. These boronic acids are prepared from 4-bromo-3-methylbenzensulfonyl chloride (CASRN72256-93-0), 4-bromo-3-chlorobenzenesulfonyl chloride (CASRN874801-46-4), 3-bromo-4-chlorobenzelsulfonyl chloride (CASRN 195201-10-6), and 3-bromo-4-methylbenzenesulfonyl chloride (CASRN 1029145-99-0), by condensation with ammonia or a primary or secondary amine, and borylating the resulting sulfonamide. One skilled in the art will appreciate that A1-2 can be converted to the boronic acid or boronate ester and coupled with an aryl or heteroaryl sulfonamide via a halide or triflate leaving group, to arrive at compounds A1-3a.

Aryl halides or triflates can be converted to boronic acids under various conditions. For example, aryl bromides can be converted to the Grignard reagent by direct insertion of magnesium in the presence of LiCl or by Mg/Br exchange with iPrMgCl/LiCl, and treating the aryl Grignard with trimethoxyborane at 0° C. (see, e.g., T. Leermann, F. R. Leroux, F. Colobert, Org. Lett., 2011, 13, 4479-4481). Aryl halides or triflates can be converted to a boronic acid employing Miyaura borylation conditions by reaction with 4,4,4′,4′,5,5,5′,5′-octamethyl-2,2′-bi(1,3,2-dioxaborolane) in the presence of a palladium catalyst such as PdCl₂(dppf) in the presence in KOAc in dioxane or DMSO at elevated temperatures (see, e.g., T. Ishiyama et al., J. Org. Chem., 1995, 60, 7508). Exemplary metal-catalyzed cross coupling reactions which can be utilized in bond formation to aryl and vinyl sp² carbon atoms include those described by Ishiyama, supra, or Suzuki, J. Organometallic Chem. 1999, 576:147-168), the Sizuki-Miyaura reaction (N. Miyaura and A. Suzuki, Chem Rev. 1995, 95, 2457-2483; A. Suzuki, J. Organometallic Chem. 1999, 576, 147-168), the Heck reaction (W. Cabri and I. Candiani, Acc. Chem. Res. 1995, 28, 2-7; A. Meijere and F. E. Meyer, Angew. Chem. Int. Ed. Eng. 1994, 33, 2379-2411) and the Stille reaction (V. Farina et al., Org. React. 1998, 50:1, 652; J. K. Stille, Angew. Chem. Int. Ed. Eng. 1986, 25, 508-524).

Oxidation of thioethers A1-3a to the corresponding sulfoxides are commonly carried out under a number of known conditions, such as reaction with aqueous solution of hydrogen peroxide, NaIO₄, tert-butylhypochlorite, acyl nitrites, sodium perborate potassium hydrogen persulfate and peracids such as peracetic acid and meta-chloroperoxybenzoic acid, to give compounds A1-3b. Typically, the sulfone can be isolated if about one equivalent of the oxidant is used. Displacement of methylsulfinic acid of A1-3b is readily accomplished by treating the sulfoxide with ammonia or the requisite substituted amine to give compounds A1-4.

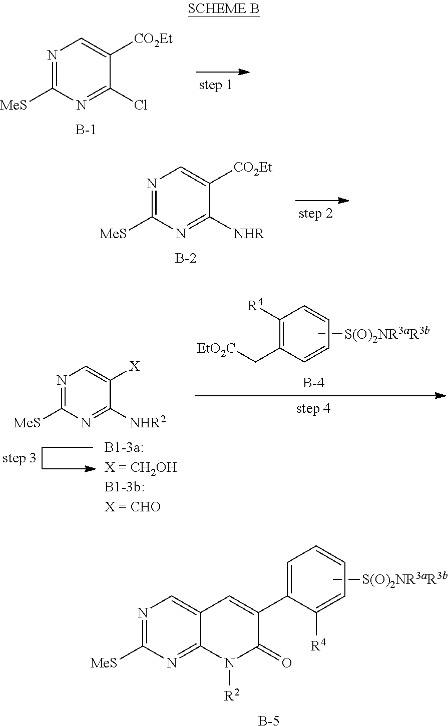

In an alternative synthesis, ethyl 4-chloro-2-(methylthio)-5-pyrimidinecarboxylate (B−1) is treated with ammonia or a substituted amine to form an amine B-2. Reduction of the pyrimidinyl ester gives the corresponding alcohol B-3a, and re-oxidation to the aldehyde affords B-3b, which can be condensed with a phenyl acetic acid derivative B-4 containing the desired sulfonamide substitution to give compounds B-5. This sequence can be adapted to acetic acid derivatives with heteroaryl substitution.

One skilled in the art will appreciate that the sequence of steps in the above reaction schemes or in the following examples can be varied without departing from the general processes disclosed herein.

### Biological Activity

Determination of the activity of PAK activity of a compound of formula I was accomplished using the PAK1 inhibition assay in Biological Example 1. Efficacy of exemplary compounds in PAK1 assays are reported in TABLE I.

### Dosage & Administration

The present invention provides pharmaceutical compositions or medicaments containing the compounds of the invention, or pharmaceutically acceptable salts thereof, and at least one therapeutically inert carrier, diluent or excipient, as well as methods of using the compounds of the invention to prepare such compositions and medicaments. In one example, compounds of formula I and pharmaceutically acceptable salts thereof with the desired degree of purity may be formulated by mixing with physiologically acceptable carriers, i.e., carriers that are non-toxic to recipients at the dosages and concentrations employed into a dosage form at ambient temperature and at the appropriate pH. The pH of the formulation depends mainly on the particular use and the concentration of compound, but typically ranges anywhere from about 3 to about 8. In one example, a compound of formula I or a pharmaceutically acceptable salt thereof is formulated in an acetate buffer, at pH 5. In another embodiment, the compounds of formula I or pharmaceutically acceptable salts thereof are sterile. The compound may be stored, for example, as a solid or amorphous composition, as a lyophilized formulation or as an aqueous solution.

Compositions are formulated, dosed, and administered in a fashion consistent with good medical practice. The term “therapeutically effective amount” denotes an amount of a compound of the present invention (or salt or free base equivalent) that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the particular disorder being treated, the severity of the disorder, the particular patient being treated, the clinical condition of the individual patient, the cause of the disorder, the site of delivery of the agent, the method of administration, the scheduling of administration, and other factors known to medical practitioners.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Sustained-release preparations may be prepared. Suitable examples of sustained-release preparations include semipermeable matrices of solid hydrophobic polymers containing a compound of formula I, or a pharmaceutically acceptable salt thereof, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A dose to treat human patients may range from about 0.1 mg to about 1000 mg of a compound of formula I, or a pharmaceutically acceptable salt thereof, or free base equivalent. A typical dose may be about 1 mg to about 300 mg of the compound, salt, or free base equivalent. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

For oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

An example of a suitable oral dosage form is a tablet containing about 25 mg, 50 mg, 100 mg, 250 mg or 500 mg of the compound of the invention (or salt or free base equivalent) compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

In one embodiment, the pharmaceutical composition also includes at least one additional anti-proliferative agent.

An embodiment, therefore, includes a pharmaceutical composition comprising a compound of formula I, or a stereoisomer or pharmaceutically acceptable salt thereof. In a further embodiment includes a pharmaceutical composition comprising a compound of formula I, or a stereoisomer or pharmaceutically acceptable salt thereof, together with a pharmaceutically acceptable carrier or excipient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The compounds of formula I, or pharmaceutically acceptable salts thereof, may be employed alone or in combination with other therapeutic agents for the treatment of a disease or disorder described herein, such as a hyperproliferative disorder (e.g., cancer). In certain embodiments, a compound of formula I, or a pharmaceutically acceptable salt thereof, is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of formula I, or a pharmaceutically acceptable salt thereof, such that they do not adversely affect each other. The combination therapy may provide “synergy” and prove “synergistic,” i.e., the effect achieved when the active ingredients used together is greater than the sum of the effects that results from using the compounds separately.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Suitable dosages for any of the above co-administered agents are those presently used and may be lowered due to the combined action (synergy) of the newly identified agent and other chemotherapeutic agents or treatments.

Combination therapies according to the present invention thus comprise the administration of at least one compound of formula I, or a stereoisomer, geometric isomer, tautomer, metabolite, or pharmaceutically acceptable salt and the use of at least one other cancer treatment method. The amounts of the compound(s) of formula I and the other pharmaceutically active chemotherapeutic agent(s) and the relative timings of administration will be selected in order to achieve the desired combined therapeutic effect.

### Articles of Manufacture

In another embodiment of the invention, an article of manufacture, or “kit,” containing materials useful for the treatment of the diseases and disorders described above is provided. In one embodiment, the kit comprises a container comprising a compound of formula I, or a stereoisomer, tautomer, or pharmaceutically acceptable salt thereof. The kit may further comprise a label or a package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of formula I, or a pharmaceutically acceptable salt thereof, or a formulation thereof which is effective for treating the condition and may have a sterile access port (for example, the container may be an intravenous solution bag or a vial having a stopper pierceable by a hypodermic injection needle). At least one active agent in the composition is a compound of formula I, or a pharmaceutically acceptable salt thereof. Alternatively, or additionally, the article of manufacture may further comprise a second container comprising a pharmaceutically diluent, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In another embodiment, the kits are suitable for the delivery of solid oral forms of a compound of formula I, or a pharmaceutically acceptable salt thereof, such as tablets or capsules. Such a kit can include a number of unit dosages. An example of such a kit is a “blister pack.” Blister packs are well known in the packaging industry and are widely used for packaging pharmaceutical unit dosage forms.

According to one embodiment, a kit may comprise (a) a first container with a compound of formula I, or a pharmaceutically acceptable salt thereof, contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### Biological Example 1

### PAK1-KD (Kinase Domain) IC₅₀ Zlyte Assay Protocol

Activity of human recombinant PAK1-KD protein may be assessed in vitro by assay of the phosphorylation of a FRET peptide substrate. Catalytically active human recombinant PAK1-KD protein is obtained by purification from Tini cells infected with a human PAK1-KD recombinant baculovirus expression vector.

The activity/inhibition of PAK1-KD was estimated by measuring the phosphorylation of a FRET peptide substrate (Ser/Thr19) labeled with coumarin and fluorescein using Z′-LYTE™ assay (Invitrogen). The peptide substrate is a consensus sequence (KKRNRRLSVA) based on various PAK substrates reported in the scientific literature. The 10 μL assay mixtures contained 50 mM HEPES (pH 7.5), 0.01% Brij-35, 10 mM MgCl₂, 1 mM EGTA, 2 μM FRET peptide substrate, and 20 pM PAK1-KD. Incubations were carried out at 22° C. in black polypropylene 384-well plates (Corning Costar). Prior to the assay, PAK1-KD, FRET peptide substrate and serially diluted test compounds were preincubated together in assay buffer (7.5 μL) for 10 minutes, and the assay was initiated by the addition of 2.5 μL assay buffer containing 160 μM ATP (4×). Following the 60-minute incubation, the assay mixtures were quenched by the addition of 5 μL of Z′-LYTETm development reagent, and 1 hour later the emissions of Coumarin (445 nm) and Fluorescein (520 nm) were determined after excitation at 400 nm using an Envision plate reader (Perkin Elmer). An emission ratio (445 nm/520 nm) was determined to quantify the degree of substrate phosphorylation.

### Referential Example 1

### (2R)-2-(p-tolylsulfonyloxymethyl)morpholine-4-carboxylate

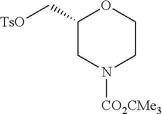

To a solution of (R)—N—BOC-2-hydroxymethylmmorpholine (4.43 g, 19.4 mmol) and N,N-dimethylaminopyridine (DMAP; 366 mg) in dichloromethane (DCM; 58.4 mL) and diisopropylethylamine (DIPEA; 3.0 equiv., 58.1 mmol, 10.3 mL) was added p-toluenesulfonyl chloride (1.3 equiv., 25.2 mmol, 4.95 g), and the reaction was kept at 25° C. for 18 h. The resulting in a brown solution was diluted with ethyl acetate (EtOAc)/diethyl ether (Et₂O) and 10% citric acid (final pH of the aqueous layer was −5). The organic layer was washed by NaHCO₃, water, and then brine, dried (MgSO₄), filtered and concentrated to afford 7.96 g of light brown solid, which was used without further purification. ¹H NMR (400 MHz, CDCl₃) δ 7.80 (d, J=8.4 Hz, 2H), 7.35 (d, J=8.0 Hz, 2H), 4.07-3.97 (m, 2H), 3.94-3.74 (m, 3H), 3.60 (dtd, J=10.3, 5.0, 2.8 Hz, 1H), 3.46 (td, J=11.6, 2.9 Hz, 1H), 2.89 (t, J=11.6 Hz, 1H), 2.67 (t, J=11.6 Hz, 1H), 2.45 (s, 3H), 1.45 (s, 9H).

### Referential Example 2

### tert-Butyl((2R,5R)-2-(2-aminoethyl)-1,3-dioxan-5-yl)carbamate (anti isomer) and tert-butyl((2S,5S)-2-(2-aminoethyl)-1,3-dioxan-5-yl)carbamate (syn isomer)

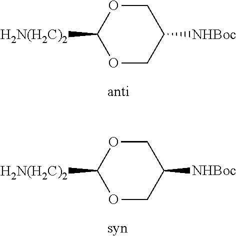

Step 1:

To a solution of 10% NaHCO₃ (aq., 3 L) was added to a solution of 3-aminopropan-1-ol (75 g, 998 mmol) in 1,4-dioxane at 0° C. Fluorenylmethyloxycarbonyl chloride (Fmoc-Cl; 309.6 g, 1.189 mol) was added dropwise to the reaction mixture. The solution was warmed to room temperature (RT) and stirred overnight. The reaction mixture was diluted with EtOAc (1 L) and H₂O (600 mL). The organic phase was separated and washed with H₂O (500 mL) and brine (500 mL), followed by drying over Na₂SO₄. The solution was concentrated in vacuo to give the crude product as a white solid. The crude product was washed with hexane (5×1 L) and dried in vacuo to afford (9H-fluoren-9-yl)methyl(3-hydroxypropyl)carbamate (313 g, 105.5%) as a white solid, which was used in the next step without further purification. ¹H-NMR (400 MHz, CDCl₃) δ 7.76 (d, J=7.5 Hz, 2H), 7.59 (d, J=7.4 Hz, 2H), 7.40 (t, J=7.2 Hz, 2H), 7.32 (td, J=8.6, 1.1 Hz, 2H), 5.03 (brs, 1H), 4.44 (d, J=6.7 Hz, 2H), 4.21 (t, J=6.6 Hz, 1H), 3.64 (t, T=5.7 Hz, 2H), 3.30-3.37 (m, 2H), 2.00-2.20 (m, 2H), 1.64-1.73 (m, 2H).

Step 2:

To a solution of N,N-dimethylsulfoxide (DMSO; 177.2 g, 2.268 mol) in anhydrous DCM (800 mL), oxalyl chloride (196.9 g, 1.549 mol) in anhydrous DCM (800 mL) was slowly added at −45° C. The reaction mixture was stirred for 20 min at −45° C. and then a solution of (9H-fluoren-9-yl)methyl(3-hydroxypropyl)carbamate (307 g, 1.032 mol) in anhydrous DCM (3.4 L) was added dropwise. After stirring for 30 min at −45° C., DIPEA (399.8 g, 3.093 mol) was added dropwise. The mixture was allowed to warm to −30° C. and stirred for another 30 min. The solvent was removed and the residue taken up in EtOAc (3 L), washed with H₂O (1 L), 5% NaHCO₃ (2×800 mL), H₂O (1 L), and brine (800 mL). The organic phase was dried (Na₂SO₄), filtered and evaporated to afford crude (9H-fluoren-9-yl)methyl(3-oxopropyl)carbamate (310 g, 104.5%) as a yellow solid. LCMS (ESI): m/z=318 [M+23]⁺.

Step 3:

p-Toluenesulfonic acid (30.6 g, 177.9 mmol) and Na₂SO₄ (2.528 kg, 17.8 mol) were added to a mixture of (9H-fluoren-9-yl)methyl(3-oxopropyl)carbamate (525 g, 1.778 mol) in toluene (6 L) and CHCl₃ (1.8 L) at RT. To the mixture was added tert-butyl(1,3-dihydroxypropan-2-yl)carbamate (251.8 g, 2.133 mol). The reaction mixture was stirred overnight at RT and floccus solid formed. The pH of the reaction mixture was adjusted to 7 by adding anhydrous Na₂CO₃ and the suspension liquid was decanted from Na₂SO₄. The floccus solid in the suspension liquid was collected by filtration and washed with DCM (2 L). The solid obtained (100 g) was retrospectively identified as {(2R,5R)-2-[2-(9H-fluoren-9-ylmethoxycarbonylamino)-ethyl]-[1,3]dioxan-5-yl}-carbamic acid tert-butyl ester (anti isomer). LCMS (ESI): m/z=491 [M+23]⁺.

The filtrates were combined and H₂O (2 L) and DCM (2 L) were added. The organic phase was separated and the aqueous phase was extracted with DCM (3×500 mL). The combined organic layers were washed with brine (1 L) and dried (Na₂SO₄), filtered and evaporated. The residue was purified by SiO₂ chromatography eluting with an EtOAc/hexane gradient (EA:hexane=1:8 to 1:4) to afford a second crop of product (390 g) as a brown oil. This crop was retrospectively identified as—9:1 mixture of {(2S,5S)-2-[2-(9H-fluoren-9-ylmethoxycarbonylamino)-ethyl]-[1,3]dioxan-5-yl}carbamic acid tert-butyl ester (syn isomer) and {(2R,5R)-2-[2-(9H-fluoren-9-ylmethoxycarbonylamino)-ethyl]-[1,3]dioxan-5-yl}-carbamic acid tert-butyl ester (anti isomer). LCMS (ESI): m/z=491 [M+23]⁺.

Step 4:

Diethylamine (1.4 L) was added to a solution of {2-[2-(9H-fluoren-9-ylmethoxy-carbonylamino)-ethyl]-[1,3]dioxan-5-yl}-carbamic acid tert-butyl ester (390 g, 832.4 mmol, the second crop from Step 3) in acetonitrile (3 L) at 0° C. The reaction mixture was stirred for 2 h at RT. The solvent was removed in vacuo and the resulting residue purified by SiO₂ column chromatography (hexane: EtOAc=1:10, then DCM:MeOH=6:1) to give tert-butyl(2-(2-aminoethyl)-1,3-dioxan-5-yl)carbamate (172.6 g, 84.2%) as a brown oil (90:10 syn:anti by ¹H-NMR). ¹H-NMR for syn isomer (400 MHz, CDCl₃) δ 5.55 (d, J=8, 5 Hz, 1H), 4.54 (t, J=4.9 Hz, 1H), 3.88-3.98 (m, 4H), 3.56 (d, J=5.1 Hz, 2H), 2.84 (t, J=6.6 Hz, 2H), 1.77-1.82 (m, 2H), 1.45 (s, 9H). LCMS (ESI): m/z=247 [M+1]⁺.

The first crop of {2-[2-(9H-fluoren-9-ylmethoxycarbonylamino)-ethyl]-[1,3]dioxan-5-yl}-carbamic acid tert-butyl ester obtained in Step 3 (100 g) was reacted in the same way as the second crop described above affording product as a white solid (33.0 g). ¹H NMR revealed this batch to be pure anti isomer. ¹H-NMR for anti isomer: (400 MHz, CDCl₃) δ 4.52 (t, J=5.0 Hz, 1H), 4.15-4.25 (m, 4H), 3.88 (brs, 1H), 3.26 (t, J=11.0 Hz, 2H), 2.81 (t, J=6.7 Hz, 2H), 1.74-1.78 (m, 2H), 1.43 (s, 9H). LCMS (ESI): m/z=247 [M+1]⁺.

### Referential Example 3

### 6-(2-chloro-4-(4-oxo-5-azaspiro[2.4]heptan-5-yl)phenyl)-8-((5,5-difluoropiperidin-3-yl)methyl)-2-(methylamino)pyrido[2,3-d]pyrimidin-7(8H)-one

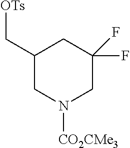

Step 1:

To a solution of 1-tert-butyl 3-methyl 5-hydroxypiperidine-1,3-dicarboxylate (2.0 g, 7.7 mmol) in DCM (20 mL) was added slowly added Dess-Martin periodinane (6.5 mg, 15.4 mmol). The mixture was stirred at RT overnight and then filtered. The filtrate was washed with H₂O and a saturated aqueous solution of Na₂CO₃. The organic layer was dried (Na₂SO₄), filtered and concentrated in vacuo and the residue was purified by SiO₂ chromatography eluting with EtOAc/petroleum ether (PE) (5:1) to afford 1-tert-butyl 3-methyl 5-oxopiperidine-1,3-dicarboxylate as colorless oil (1.2 g, 61%). LCMS (ESI): m/z=202.0[[M+1]-56]⁺.

Step 2:

To a solution of 1-tert-butyl 3-methyl 5-oxopiperidine-1,3-dicarboxylate (1.1 g, 4.06 mmol) in DCM (20 mL) cooled to −78° C. was added dropwise diethylaminosulfur trifluoride (2.0 g, 12.18 mmol). The mixture was stirred at RT for overnight and then H₂O and DCM (30 mL) were added. The organic layer was dried (Na₂SO₄), filtered and concentrated in vacuo. The residue was purified by SiO₂ chromatography eluting with EtOAc/PE (8:1) to afford 1-tert-butyl 3-methyl 5,5-difluoropiperidine-1,3-dicarboxylate as colorless oil (800 mg, 67%). LCMS (ESI): m/z=224.1 [[M+l]-56]⁺.

Step 3:

To a solution of 1-tert-butyl 3-methyl 5,5-difluoropiperidine-1,3-dicarboxylate (800 mg, 650 mmol) in DCM (10 mL) at 0° C. was added slowly NaBH₄ (650 mg, 17.2 mmol). The mixture was stirred at RT overnight and then concentrated in vacuo. The residue was diluted with EtOAc (50 mL) and H₂O (50 mL) the organic layer was dried (Na₂SO₄), filtered and concentrated in vacuo to afford tert-butyl 3,3-difluoro-5-(hydroxymethyl)piperidine-1-carboxylate as colorless oil (1.2 g, crude). LCMS (ESI): m/z=196.1 [[M+1]-56]⁺.

Step 4:

To a solution of tert-butyl 3,3-difluoro-5-(hydroxymethyl)piperidine-1-carboxylate (1.2 g, 4.78 mmol) in DCM (10 mL) was added p-toluenesulfonyl chloride (1.1 g, 5.73 mmol), triethylamine (TEA; 1.45 g, 14.3 mmol) and DMAP (58 mg, 0.478 mmol). The mixture was stirred at RT overnight and the solution was then concentrated in vacuo. The residue was purified by SiO₂ chromatography eluting with EtOAc/PE (3:1) to afford tert-butyl 3,3-difluoro-5-(tosyloxymethyl)piperidine-1-carboxylate as white solid (1.2 g, 62%). LCMS (ESI): m/z=350.1 [[M+1]-56]⁺.

### Example 1

### Compound I-2

### N-Ethyl-4-(8-ethyl-2-((2-(1-methylpiperidin-4-yl)ethyl)amino)-7-oxo-7,8-dihydropyrido[2,3-d]pyrimidin-6-yl)-3-methylbenzenesulfonamide (I-2)

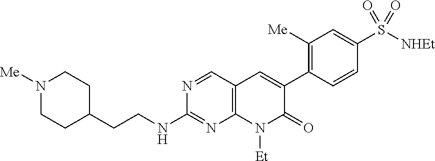

Step 1:

A 20-mL microwave vial was charged with 6-bromo-8-ethyl-2-methylsulfanyl-pyrido[2,3-d]pyrimidin-7-one (1.0 equiv, 1.333 mmol, 400.0 mg), [4-(ethylsulfamoyl)-2-methyl-phenyl]boronic acid (1.2 equiv, 1.599 mmol, 388.7 mg), dibasic potassium phosphate (3.0 equiv, 3.998 mmol, 696.3 mg), (dppf)Cl₂Pd(II) (0.06 equiv, 0.07995 mmol, 59.09 mg), degassed DMF (4.935 mL), and degassed 1,4-dioxane (4.935 mL). The reaction mixture was thrice vacuum purged and back-filled with N₂. The vial was capped, and the reaction mixture was irradiated in a microwave at 120° C. for 40 min. The reaction mixture was diluted with EtOAc then filtered through a pad of diatomaceous earth. The organic layer was washed with water and brine, dried over Na₂SO₄, filtered, and concentrated, then purified by SiO₂ chromatography eluting with 10 to 100% EtOAc in heptane to afford 330 mg of N-ethyl-4-(8-ethyl-2-methylsulfanyl-7-oxo-pyrido[2,3-d]pyrimidin-6-yl)-3-methyl-benzenesulfonamide as a beige foam. ¹H NMR (400 MHz, CDCl₃) δ 8.66 (d, J=1.4 Hz, 1H), 7.78 (d, J=1.8 Hz, 1H), 7.74 (dd, J=7.9, 2.0 Hz, 1H), 7.57 (d, J=1.2 Hz, 1H), 7.37 (d, J=7.9 Hz, 1H), 4.55 (dd, J=7.7, 6.5 Hz, 2H), 4.25 (t, J=6.1 Hz, 1H), 3.13-3.01 (m, 2H), 2.67 (d, J=1.3 Hz, 3H), 2.30 (s, 3H), 1.41-1.33 (m, 3H), 1.16 (td, J=7.2, 1.3 Hz, 3H).

Step 2:

To N-ethyl-4-(8-ethyl-2-methylsulfanyl-7-oxo-pyrido[2,3-d]pyrimidin-6-yl)-3-methyl-benzenesulfonamide (1.0 equiv, 0.2473 mmol, 103.5 mg) in anhydrous DCM (7.926 mL) at 0° C. was added portion-wise m-chloroperoxybenzoic acid (MCPBA; 1.1 equiv, 0.2720 mmol, 60.97 mg). The reaction mixture was stirred at 0° C. for 2 h, then at RT for 2 h. The organics were washed with water and brine, dried over Na₂SO₄, filtered, and concentrated to afford 105.7 mg of N-ethyl-4-(8-ethyl-2-methylsulfinyl-7-oxo-pyrido[2,3-d]pyrimidin-6-yl)-3-methyl-benzenesulfonamide as a solid. The material was used in the next step without further purification.

Step 3:

A mixture of N-ethyl-4-(8-ethyl-2-methylsulfinyl-7-oxo-pyrido[2,3-d]pyrimidin-6-yl)-3-methyl-benzenesulfonamide (1.0 equiv, 0.2433 mmol, 105.7 mg), 2-(1-methyl-4-piperidyl)ethanamine (1.3 equiv, 0.3162 mmol, 44.98 mg), and DIPEA (4.0 equiv, 0.9731 mmol, 125.8 mg, 0.170 mL) in anhydrous THF (1.98 mL) was stirred at RT under nitrogen for 22 h. The reaction mixture was concentrated and purified by reverse-phase HPLC to afford 80.3 mg of 1-2. LC-MS m/z: 513.3 mg [M+1]⁺. ¹H NMR (400 MHz, DMSO-d₆) δ 8.61 (s, 1H), 7.94 (br s, 1H), 7.74 (s, 1H), 7.67 (d, J=1.9 Hz, 1H), 7.63 (dd, J=8.0, 1.9 Hz, 1H), 7.56-7.49 (m, 1H), 7.40 (d, J=7.9 Hz, 1H), 4.39-4.28 (m, 2H), 3.40 (q, J=6.7 Hz, 2H), 2.82 (p, J=7.0 Hz, 2H), 2.72 (d, J=11.3 Hz, 2H), 2.23 (s, 3H), 2.12 (s, 3H), 1.86-1.75 (m, 2H), 1.67 (d, J=12.7 Hz, 2H), 1.59-1.45 (m, 2H), 1.35-1.10 (m, 6H), 1.02 (t, J=7.2 Hz, 3H).

### Example 2

### Compound I-3

### 4-(8-Ethyl-2-((2-(1-methylpiperidin-4-yl)ethyl)amino)-7-oxo-7,8-dihydropyrido[2,3-d]pyrimidin-6-yl)-N,N,3-trimethylbenzenesulfonamide (I-3)

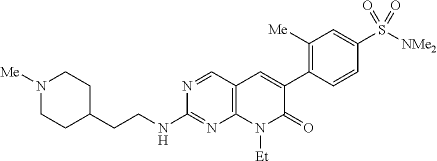 [SMILES: CCn1c(=O)c(-c2ccc(S(=O)(=O)N(C)C)cc2C)cc2cnc(NCCC3CCN(C)CC3)nc21]

Step 1:

A 20-mL microwave vial was charged with 6-bromo-8-ethyl-2-methylsulfanyl-pyrido[2,3-d]pyrimidin-7-one (1.0 equiv, 1.333 mmol, 400.0 mg), [4-(dimethylsulfamoyl)-2-methyl-phenyl]boronic acid (1.2 equiv, 1.599 mmol, 388.7 mg), dibasic potassium phosphate (3.0 equiv, 3.998 mmol, 696.3 mg), (dppf)Cl₂Pd(II) (0.06 equiv, 0.07995 mmol, 59.09 mg), degassed DMF (4.935 mL), and degassed 1,4-dioxane (4.935 mL). The reaction mixture was thrice vacuum purged and filled with N₂. The vial was capped, and the reaction mixture was irradiated in a microwave at 120° C. for 40 min. The reaction mixture was diluted with EtOAc then filtered through a pad of diatomaceous earth. The organic layer was washed with water and brine, dried over Na₂SO₄, filtered, and concentrated, then purified by SiO₂ chromatography eluting with 10 to 100% EtOAc in heptane to afford 260 mg of 4-(8-ethyl-2-methylsulfanyl-7-oxo-pyrido[2,3-d]pyrimidin-6-yl)-N,N,3-trimethyl-benzenesulfonamide as a beige foam. ¹H NMR (400 MHz, CDCl₃) δ 8.67 (d, J=1.5 Hz, 1H), 7.72-7.62 (m, 2H), 7.59 (d, J=1.6 Hz, 1H), 7.39 (d, J=7.9 Hz, 1H), 4.56 (q, J=7.0 Hz, 2H), 2.77 (d, J=1.4 Hz, 6H), 2.67 (d, J=1.4 Hz, 3H), 2.32 (s, 3H), 1.41-1.33 (m, 3H).

Step 2:

To a solution of 4-(8-ethyl-2-methylsulfanyl-7-oxo-pyrido[2,3-d]pyrimidin-6-yl)-N,N,3-trimethyl-benzenesulfonamide (1.0 equiv, 0.177 mmol, 74.0 mg) in anhydrous DCM (5.67 mL) at 0° C. was added portion-wise MCPBA (1.10 equiv, 0.195 mmol, 43.6 mg). The reaction mixture was stirred at 0° C. for 2 h, then at RT for 2 h. The reaction was quenched by adding sat′d. NaHCO₃ then extracted with EtOAc (3 times). The organics were washed with water and brine, dried over Na₂SO₄, filtered, and concentrated to afford 75.5 mg of 4-(8-ethyl-2-methylsulfinyl-7-oxo-pyrido[2,3-d]pyrimidin-6-yl)-N,N,3-trimethyl-benzenesulfonamide as a solid which was used without further purification.

Step 3:

A mixture of 4-(8-ethyl-2-methylsulfinyl-7-oxo-pyrido[2,3-d]pyrimidin-6-yl)-N,N,3-trimethyl-benzenesulfonamide (1.0 equiv, 0.1738 mmol, 75.5 mg), 2-(1-methyl-4-piperidyl)ethanamine (1.3 equiv, 0.2259 mmol, 32.13 mg), and DIPEA (4.0 equiv, 0.6951 mmol, 89.83 mg, 0.121 mL) in anhydrous THF (1.441 mL) was stirred at RT under nitrogen for 17 h. The reaction mixture was concentrated and purified by reverse-phase HPLC to afford 52.7 mg of target compound. LC-MS m/z: 513.3 mg [M+1]⁺. ¹H NMR (400 MHz, d₆-DMSO) δ 8.61 (s, 1H), 8.00-7.91 (m, 1H), 7.76 (s, 1H), 7.64 (d, J=1.9 Hz, 1H), 7.59 (dd, J=8.0, 2.0 Hz, 1H), 7.46 (d, J=8.0 Hz, 1H), 4.35 (q, J=7.2 Hz, 2H), 3.40 (q, J=6.9 Hz, 2H), 2.72 (d, J=11.1 Hz, 2H), 2.66 (s, 6H), 2.26 (s, 3H), 2.12 (s, 3H), 1.80 (t, J=11.2 Hz, 2H), 1.71-1.60 (m, 2H), 1.54 (q, J=7.1 Hz, 2H), 1.36-1.10 (m, 6H).

### Example 3

### Compound I-6

### N-Cyclopropyl-4-(8-ethyl-2-((2-(1-methylpiperidin-4-yl)ethyl)amino)-7-oxo-7,8-dihydropyrido[2,3-d]pyrimidin-6-yl)-3-methylbenzenesulfonamide (I-6)

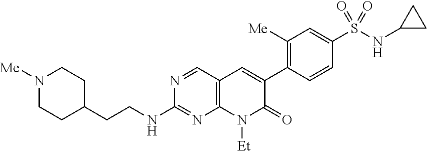 [SMILES: CCn1c(=O)c(-c2ccc(S(=O)(=O)NC3CC3)cc2C)cc2cnc(NCCC3CCN(C)CC3)nc21]

Step 1:

A 20-mL microwave vial was charged with 6-bromo-8-ethyl-2-methylsulfanyl-pyrido[2,3-d]pyrimidin-7-one (1.0 equiv, 0.9994 mmol, 300.0 mg), [4-(cyclopropylsulfamoyl)-2-methyl-phenyl]boronic acid, (CASRN1217501-49-9; 1.50 equiv, 1.499 mmol, 382.4 mg), dibasic potassium phosphate (3.0 equiv, 2.998 mmol, 522.2 mg), (dppf)Cl₂Pd(II) (0.06 equiv, 0.05996 mmol, 44.32 mg), degassed DMF (3.701 mL), and degassed 1,4-dioxane (3.701 mL). The reaction mixture was thrice vacuum purged and refilled with N₂. The vial was capped, and the reaction mixture was irradiated in a microwave at 120° C. for 40 min. The reaction mixture was diluted with EtOAc then filtered through a pad of diatomaceous earth. The organic layer was washed with water and brine, dried over Na₂SO₄, filtered, and concentrated, then purified SiO₂ chromatography eluting with 20 to 100% EtOAc in heptane to afford 290 mg of N-cyclopropyl-4-(8-ethyl-2-methylsulfanyl-7-oxo-pyrido[2,3-d]pyrimidin-6-yl)-3-methyl-benzenesulfonamide as a beige foam. ¹H NMR (400 MHz, CDCl₃) δ 8.66 (d, J=1.1 Hz, 1H), 7.82 (d, J=1.8 Hz, 1H), 7.81-7.75 (m, 1H), 7.58 (d, J=1.1 Hz, 1H), 7.38 (d, J=8.0 Hz, 1H), 4.80 (s, 1H), 4.56 (q, J=6.9 Hz, 2H), 2.67 (d, J=1.0 Hz, 3H), 2.35-2.27 (m, 4H), 1.43-1.35 (m, 3H), 0.72-0.61 (m, 4H).

Step 2:

To a solution of N-cyclopropyl-4-(8-ethyl-2-methylsulfanyl-7-oxo-pyrido[2,3-d]pyrimidin-6-yl)-3-methyl-benzenesulfonamide (1.0 equiv, 0.1626 mmol, 70.00 mg) in anhydrous DCM (5.2 mL) at 0° C. was added portionwise MCPBA (1.1 equiv, 0.1789 mmol, 40.09 mg). The reaction mixture was stirred at 0° C. for 1 h. The reaction was quenched by adding sat′ d. NaHCO₃ then extracted with EtOAc (3 times). The organics were washed with water and brine, dried over Na₂SO₄, filtered, and concentrated to afford 64.4 mg of N-cyclopropyl-4-(8-ethyl-2-methylsulfinyl-7-oxo-pyrido[2,3-d]pyrimidin-6-yl)-3-methyl-benzenesulfonamide as a solid which was used in the next step without further purification.

Step 3:

A mixture of N-cyclopropyl-4-(8-ethyl-2-methylsulfinyl-7-oxo-pyrido[2,3-d]pyrimidin-6-yl)-3-methyl-benzenesulfonamide (1.0 equiv, 0.1442 mmol, 64.4 mg), 2-(1-methyl-4-piperidyl)ethanamine (1.3 equiv, 0.1875 mmol, 26.67 mg), and DIPEA (4.0 equiv, 0.5769 mmol, 74.57 mg, 0.101 mL) in anhydrous THF (1.17 mL) was stirred at RT under nitrogen for 4.5 h. The reaction mixture was concentrated and purified by reverse-phase HPLC to afford 44.4 mg of I-6. LC-MS m/z: 525.3 [M+1]⁺.

### Example 4

### Compound I-5

### 8-Ethyl-6-(2-methyl-4-(pyrrolidin-1-ylsulfonyl)phenyl)-2-((2-(1-methylpiperidin-4-yl)ethyl)amino)pyrido[2,3-d]pyrimidin-7(8H)-one (I-5)

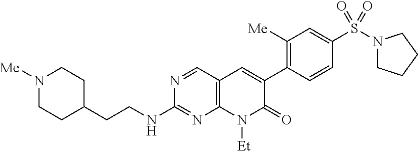 [SMILES: CCn1c(=O)c(-c2ccc(S(=O)(=O)N3CCCC3)cc2C)cc2cnc(NCCC3CCN(C)CC3)nc21]

Step 1:

A 20-mL microwave vial was charged with 6-bromo-8-ethyl-2-methylsulfanyl-pyrido[2,3-d]pyrimidin-7-one (1.0 equiv, 0.9994 mmol, 300.0 mg), (2-methyl-4-pyrrolidin-1-ylsulfonyl-phenyl)boronic acid (CASRN 1217501-51-3; 1.50 equiv, 1.499 mmol, 403.4 mg), dibasic potassium phosphate (3.0 equiv, 2.998 mmol, 522.2 mg), (dppf)Cl₂Pd(II) (0.06 equiv, 0.05996 mmol, 44.32 mg), degassed DMF (3.701 mL), and degassed 1,4-dioxane (3.701 mL). The reaction mixture was thrice vacuum purged and refilled with N₂. The vial was capped, and the reaction mixture was irradiated in microwave at 120° C. for 40 min. The reaction mixture was diluted with EtOAc then filtered through a pad of diatomaceous earth. The organic layer was washed with water and brine, dried over Na₂SO₄, filtered, and concentrated, then purified by SiO₂ chromatography eluting with 10 to 100% EtOAc in heptane to afford 234.9 mg of 8-ethyl-6-(2-methyl-4-pyrrolidin-1-ylsulfonyl-phenyl)-2-methylsulfanyl-pyrido[2,3-d]pyrimidin-7-one as a beige foam. ¹H NMR (400 MHz, CDCl₃) δ 8.66 (s, 1H), 7.74 (s, 1H), 7.73-7.68 (m, 1H), 7.58 (s, 1H), 7.37 (d, J=8.0 Hz, 1H), 4.56 (q, J=7.0 Hz, 2H), 3.35-3.25 (m, 4H), 2.67 (s, 3H), 2.31 (s, 3H), 1.87-1.76 (m, 4H), 1.38 (t, J=7.0 Hz, 3H).

Step 2:

A solution of 8-ethyl-6-(2-methyl-4-pyrrolidin-1-ylsulfonyl-phenyl)-2-methylsulfanyl-pyrido[2,3-d]pyrimidin-7-one (1.0 equiv, 0.1979 mmol, 88.00 mg) in anhydrous DCM (6.3 mL) at 0° C. was added portion-wise MCPBA (1.1 equiv, 0.2177 mmol, 48.79 mg). The reaction mixture was stirred at 0° C. for 1 h. The reaction was quenched by adding sat′d. NaHCO₃ then extracted with EtOAc (3 times). The organics were washed with water and brine, dried over Na₂SO₄, filtered, and concentrated to afford 89.9 mg of 8-ethyl-6-(2-methyl-4-pyrrolidin-1-ylsulfonyl-phenyl)-2-methylsulfinyl-pyrido[2,3-d]pyrimidin-7-one as a solid which was used in the next step without further purification.

Step 3:

A mixture of 8-ethyl-6-(2-methyl-4-pyrrolidin-1-ylsulfonyl-phenyl)-2-methylsulfinyl-pyrido[2,3-d]pyrimidin-7-one (1.0 equiv, 0.193 mmol, 88.9 mg), 2-(1-methyl-4-piperidyl)ethanamine (1.3 equiv, 0.251 mmol, 35.7 mg), and DIPEA (4.0 equiv, 0.772 mmol, 99.8 mg, 0.135 mL) in anhydrous THF (1.57 mL) was stirred at RT under nitrogen for 17 h. The reaction mixture was concentrated and purified by reverse-phase HPLC to afford 60.2 mg of target compound. LC-MS m/z: 539.3 [M+1]⁺. ¹H NMR (400 MHz, DMSO-d₆) δ 8.61 (s, 1H), 7.99-7.92 (m, 1H), 7.76 (s, 1H), 7.69 (d, J=1.9 Hz, 1H), 7.64 (dd, J=7.9, 2.0 Hz, 1H), 7.44 (d, J=7.9 Hz, 1H), 4.39-4.26 (m, 2H), 3.40 (q, J=6.9 Hz, 2H), 3.24-3.12 (m, 4H), 2.72 (d, J=11.1 Hz, 2H), 2.25 (s, 3H), 2.12 (s, 3H), 1.80 (t, J=11.4 Hz, 2H), 1.74-1.61 (m, 6H), 1.59-1.45 (m, 2H), 1.35-1.12 (m, 6H).

### Example 5

### Compound I-4

### 8-Ethyl-6-(2-methyl-4-(morpholinosulfonyl)phenyl)-2-((2-(1-methylpiperidin-4-yl)ethyl)amino)pyrido[2,3-d]pyrimidin-7(8H)-one (I-4)

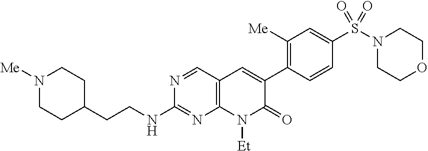 [SMILES: CCn1c(=O)c(-c2ccc(S(=O)(=O)N3CCOCC3)cc2C)cc2cnc(NCCC3CCN(C)CC3)nc21]

Step 1:

A 20-mL microwave vial was charged with 6-bromo-8-ethyl-2-methylsulfanyl-pyrido[2,3-d]pyrimidin-7-one (1.0 equiv, 0.9994 mmol, 300.0 mg), (2-methyl-4-morpholinosulfonyl-phenyl)boronic acid (CASRN 1217501-53-5; 1.50 equiv, 1.499 mmol, 427.4 mg), dibasic potassium phosphate (3.0 equiv, 2.998 mmol, 522.2 mg), (dppf)Cl₂Pd(II) (0.10 equiv, 0.09994 mmol, 73.86 mg), degassed DMF (3.7 mL), and degassed 1,4-dioxane (3.7 mL). The reaction mixture was thrice vacuum purged and refilled with N₂. The vial was capped, and the reaction mixture was irradiated in microwave at 120° C. for 40 min. The reaction mixture was diluted with EtOAc then filtered through a pad of diatomaceous earth. The organic layer was washed with water and brine, dried over Na₂SO₄, filtered, and concentrated, then purified by SiO₂ chromatography eluting with 20 to 100% EtOAc in heptane to afford 255.3 mg of 8-ethyl-6-(2-methyl-4-morpholinosulfonyl-phenyl)-2-methylsulfanyl-pyrido[2,3-d]pyrimidin-7-one as a beige foam. LC-MS m/z: 461.0 [M+1]⁺. ¹H NMR (400 MHz, CDCl₃) δ 8.67 (s, 1H), 7.68-7.61 (m, 2H), 7.59 (s, 1H), 7.40 (d, J=7.8 Hz, 1H), 4.56 (q, J=7.0 Hz, 2H), 3.79-3.72 (m, 4H), 3.10-3.04 (m, 4H), 2.67 (s, 3H), 2.32 (s, 3H), 1.38 (t, J=7.1 Hz, 3H).

Step 2:

To a solution of 8-ethyl-6-(2-methyl-4-morpholinosulfonyl-phenyl)-2-methylsulfanyl-pyrido[2,3-d]pyrimidin-7-one (1.0 equiv, 0.1563 mmol, 72.00 mg) in anhydrous DCM (5.0 mL) at 0° C. was added portion-wise MCPBA (1.1 equiv, 0.1719 mmol, 38.54 mg). The reaction mixture was stirred at 0° C. for 1 h. The reaction was quenched by adding sat′d. NaHCO₃ then extracted with EtOAc (3 times). The organics were washed with water and brine, dried over Na₂SO₄, filtered, and concentrated to afford 73.8 mg of 8-ethyl-6-(2-methyl-4-morpholinosulfonyl-phenyl)-2-methylsulfinyl-pyrido[2,3-d]pyrimidin-7-one as a solid which was used in the next step without further purification.

Step 3:

A mixture of 8-ethyl-6-(2-methyl-4-morpholinosulfonyl-phenyl)-2-methylsulfinyl-pyrido[2,3-d]pyrimidin-7-one (1.0 equiv, 0.1548 mmol, 73.80 mg), 2-(1-methyl-4-piperidyl)ethanamine (1.3 equiv, 0.2013 mmol, 28.63 mg), and DIPEA (4.0 equiv, 0.6194 mmol, 80.05 mg, 0.108 mL) in anhydrous THF (1.26 mL) was stirred at RT under nitrogen for 24 h. The reaction mixture was concentrated and purified by reverse-phase HPLC to afford 49.6 mg of target compound. LC-MS m/z: 555.3 [M+1]⁺. ¹H NMR (400 MHz, d₆-DMSO) δ 8.61 (s, 1H), 7.96 (t, J=5.6 Hz, 1H), 7.78 (s, 1H), 7.63 (d, J=1.9 Hz, 1H), 7.59 (dd, J=8.0, 2.0 Hz, 1H), 7.48 (d, J=8.0 Hz, 1H), 4.40-4.28 (m, 2H), 3.65 (dd, J=5.9, 3.5 Hz, 4H), 3.41 (q, J=6.9 Hz, 2H), 2.98-2.85 (m, 4H), 2.72 (d, J=11.3 Hz, 2H), 2.27 (s, 3H), 2.12 (s, 3H), 1.80 (t, J=11.3 Hz, 2H), 1.67 (d, J=12.4 Hz, 2H), 1.59-1.46 (m, 2H), 1.33-1.11 (m, 6H).

### Example 6

### Compound I-1

### 3-(8-Ethyl-2-((2-(1-methylpiperidin-4-yl)ethyl)amino)-7-oxo-7,8-dihydropyrido[2,3-d]pyrimidin-6-yl)-N,N-dimethylbenzenesulfonamide (I−1)

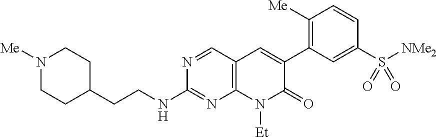 [SMILES: CCn1c(=O)c(-c2cc(S(=O)(=O)N(C)C)ccc2C)cc2cnc(NCCC3CCN(C)CC3)nc21]

A 5-mL microwave vial was charged 6-bromo-8-ethyl-2-[2-(1-methyl-4-piperidyl)ethylamino]pyrido[2,3-d]pyrimidin-7-one (1.0 equiv, 0.1522 mmol, 60.00 mg), [3-(dimethylsulfamoyl)phenyl]boronic acid (2.0 equiv, 0.3043 mmol, 69.72 mg), dibasic potassium phosphate (3.2 equiv, 0.4869 mmol, 84.81 mg), (dppf)Cl₂Pd(II) (0.75 equiv, 0.1141 mmol, 84.35 mg), degassed DMF (1.5 mL), and degassed 1,4-dioxane (1.5 mL). The reaction mixture was thrice vacuum purged and refilled with N₂. The vial was capped and the reaction mixture was irradiated in microwave at 120° C. for 45 min. The reaction mixture was diluted with EtOAc then filtered through a pad of diatomaceous earth. The organic layer was washed with water and brine, dried over Na₂SO₄, filtered, concentrated and purified by reverse-phase HPLC to afford 49.3 mg of target compound. LC-MS m/z: 499.3 [M+1]⁺. ¹H NMR (400 MHz, DMSO-d₆) δ 8.68 (s, 1H), 8.21 (s, 1H), 8.09 (d, J=9.0 Hz, 2H), 8.04-7.96 (m, 2H), 7.72-7.69 (m, 1H), 4.37 (q, J=7.8, 7.3 Hz, 2H), 3.41 (q, J=6.9 Hz, 2H), 2.83 (d, J=11.2 Hz, 2H), 2.66 (s, 6H), 2.22 (s, 3H), 1.99 (t, J=11.6 Hz, 2H), 1.71 (d, J=12.6 Hz, 2H), 1.55 (q, J=7.1 Hz, 2H), 1.34 (br s, 1H), 1.29-1.15 (m, 5H).

Compounds I-7 to I-14 were prepared using methods analogous to those described above. Satisfactory analytical data were obtained for each compound.

| Compound | LC/MS (observed) |
|---|---|
| I-7 | 499.3 |
| I-8 | 533.3 |
| I-9 | 436 |
| I-10 | 506.2 |
| I-11 | 408.2 |
| I-12 | 436 |
| I-13 | 533.2 |
| I-14 | 533.1 |

The additional exemplary compounds shown above in Table IA may be prepared using methods analogous to those described above.

### Example 7

Pharmaceutical compositions of the subject Compounds for administration via several routes can be prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |

| | |
|---|---|
| Distilled water | q.s. to 100 ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound of formula IA: 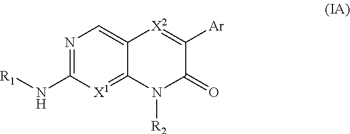
wherein:
X¹ and X² are independently CH or N;
Ar is phenyl, pyridinyl, pyridine-N-oxide, pyridinone, pyrimidinyl, pyridazinyl, or pyrazinyl, each substituted by —S(═O)ₙR³, and optionally further substituted by one or two groups independently selected from C₁₋₆ alkyl, C₁₋₆ alkoxy, C₁₋₆ haloalkyl, C₃₋₆ cycloalkyl, halogen, and cyano;
wherein n is 0, 1, or 2; and
R³ is NR^{3a}R^{3b};
wherein R^{3a} and R^{3b} are independently selected from:
(a) hydrogen;
(b) C₁₋₁₀ alkyl;
(c) C₁₋₆ haloalkyl; and
(d) C₃₋₆ cycloalkyl;
wherein any said alkyl or said cycloalkyl is optionally substituted either with one or two hydroxyl groups or with a NR³CR^{3d} group;
wherein R³ and R^{3d} are independently hydrogen or C₁₋₃ alkyl; or R^{1c} and R^{3d} together with the nitrogen to which they are attached form a cyclic amine; or
(e) R^{3a} and R^{3b} together with the nitrogen atom to which they are attached form a cyclic amine, optionally substituted with one or two groups selected from C₁₋₄ alkyl and oxo;
R¹ is (alkylene)₀₋₆R^{1a};
wherein R^{1a} is (i) hydrogen, (ii) hydroxyl, (iii) C₁₋₆ alkoxy, (iv) NR^{1b}R^{1c}, or (v) a heterocycle selected from the group consisting of azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, N—C₁₋₆ alkyl piperazinyl, tetrahydropyranyl, tetrahydrofuranyl, 3-azabicyclo[3.1.0]hexan-6-yl, and 5-amino-1,3-dioxolan-2-yl, each said heterocycle optionally substituted by oxo, hydroxyl, amino, C₁₋₃ alkylamino, or C₁₋₃ dialkylamino;
wherein R^{1b} and R^{1c} are independently hydrogen or C₁₋₃ alkyl optionally substituted by OH or NR^{1d}R^{1e}; or R^{1b} and R^{1c} together with the nitrogen to which they are attached form a cyclic amine optionally substituted either with one or two hydroxyl groups or with a NR^{3c}R^{3d} group; wherein R^{1d} and R^{1e} are independently hydrogen or C₁₋₃ alkyl; and
with the proviso that when R^{1a} is NR^{1b}R^{1c} or OH, the alkylene moiety of R¹ contains at least contains two carbons;
R² is selected from the group consisting of:
(i) hydrogen;
(ii) C₁₋₆ alkyl;
(iii) C₃₋₇ cycloalkyl;
(iv) (alkylene)₁₋₃NR^{a}R^{b}, wherein the alkylene chain is optionally substituted by a hydroxyl;
(v) (alkylene)₁₋₃OR⁵, wherein R⁵ is (alkylene)₂₋₄NR^{a}R^{b} or a heterocycle selected from azetidine, pyrrolidine, piperidine, and azepane;
(vi) (alkylene)₀₋₃-(C₄₋₆-cycloalkyl-NR^{a}R^{b});
(vii) (alkylene)₀₋₃-heterocyclyl, wherein heterocyclyl refers to azetidinyl, pyrrolidinyl, piperidinyl, azepanyl, morpholinyl, 1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl, 5,8-dioxa-2-azaspiro[3.4]octan-6-yl, 5,5-dioxido-5-thia-2-azaspiro[3.4]octan-7-yl, thiomorpholinyl, or piperazinyl, each optionally substituted by one or more moieties selected from the group consisting of C₁₋₆ alkyl, C(═O)CHR^{f}NH₂, halogen, oxo, hydroxyl, amino, C₁₋₃ alkylamino, C₁₋₃ dialkylamino, and C₁₋₆-hydroxyalkyl;
wherein R^{a} and R^{b} are independently hydrogen, C₁₋₃ alkyl, or C₂₋₄ hydroxyalkyl; or R^{a} and R^{b} together with the nitrogen to which they are attached form a cyclic amine optionally substituted either with one or two hydroxyl groups or with a NR^{3c}R^{3d} group; and
R^{f} is hydrogen or C₁₋₃ alkyl;
or a pharmaceutically acceptable salt thereof.

2. The compound of claim 1, or a pharmaceutically acceptable salt thereof, wherein X¹ is N and X² is CH.

3. The compound of claim 1, or a pharmaceutically acceptable salt thereof, wherein Ar is phenyl, pyridinyl, or pyrimidinyl, each substituted by —S(═O)ₙR³, and optionally further substituted by one or two groups independently selected from C₁₋₆ alkyl, C₁₋₆ alkoxy, C₁₋₆ haloalkyl, C₃₋₆ cycloalkyl, halogen, and cyano.

4. The compound of claim 1, or a pharmaceutically acceptable salt thereof, wherein Ar is: 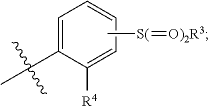
wherein R⁴ is C₁₋₃ alkyl, bromo, chloro, fluoro, cyano, OMe, or CF₃.

5. The compound of claim 1, or a pharmaceutically acceptable salt thereof, wherein Ar is: 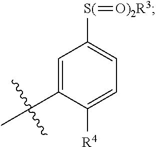
wherein R⁴ is C₁₋₃ alkyl, bromo, chloro, fluoro, cyano, OMe, or CF₃.

6. The compound of claim 1, or a pharmaceutically acceptable salt thereof, wherein Ar is: 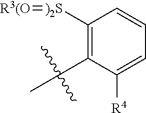
wherein R⁴ is C₁₋₃ alkyl, bromo, chloro, fluoro, cyano, OMe, or CF₃.

7. The compound of claim 1, or a pharmaceutically acceptable salt thereof, wherein Ar is: 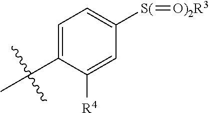
wherein R⁴ is C₁₋₃ alkyl, bromo, chloro, fluoro, cyano, OMe, or CF₃.

8. The compound of claim 4, or a pharmaceutically acceptable salt thereof, wherein R⁴ is methyl or chloro.

9. The compound of claim 1, or a pharmaceutically acceptable salt thereof, wherein R^{3a} and R^{3b} are independently hydrogen, C₁₋₄ alkyl (optionally substituted with hydroxyl), C₁₋₄ haloalkyl, or C₃₋₆ cycloalkyl, or R^{3a} and R^{3b} taken together with the nitrogen to which they are attached form a cyclic amine optionally substituted with C₁₋₄ alkyl or two oxo groups.

10. The compound of claim 1, or a pharmaceutically acceptable salt thereof, wherein R¹ is C₁₋₃ alkyl.

11. The compound of claim 1, or a pharmaceutically acceptable salt thereof, wherein R¹ is (alkylene)₀₋₆R^{1a}, wherein R^{1a} is heterocycle selected from the group consisting of azetidinyl, pyrrolidinyl, piperidinyl, N—C₁₋₆ alkyl azetidinyl, N—C₁₋₆ alkyl pyrrolidinyl, N—C₁₋₆ alkyl piperidinyl, N—C₁₋₆ alkyl piperazinyl, tetrahydropyranyl, tetrahydrofuranyl, and 5-amino-1,3-dioxolan-2-yl, each said heterocycle optionally substituted by oxo, hydroxyl, amino, C₁₋₃ alkylamino, or C₁₋₃ dialkylamino.

12. The compound of claim 1, or a pharmaceutically acceptable salt thereof, wherein R¹ is (alkylene)₂₋₄R^{1a}.

13. The compound of claim 1, or a pharmaceutically acceptable salt thereof, wherein R¹ is selected from the group consisting of:

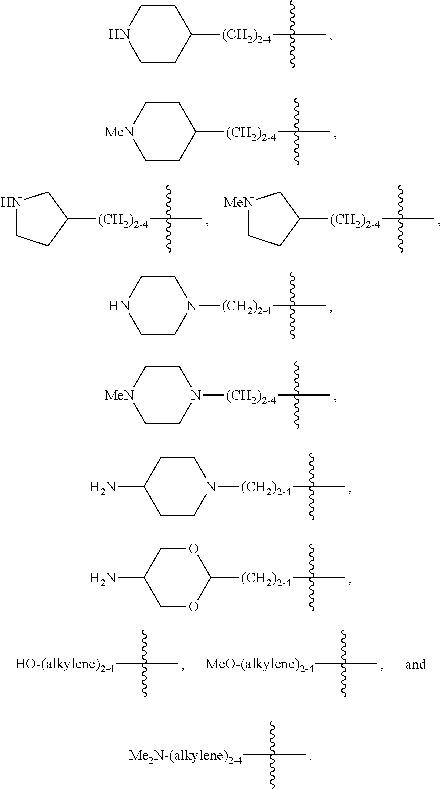

14. The compound of claim 1, or a pharmaceutically acceptable salt thereof, wherein R² is H, C₁₋₆ alkyl, or C₃₋₇ cycloalkyl.

15. The compound of claim 1, or a pharmaceutically acceptable salt thereof, wherein R² is selected from the group consisting of 1-morpholin-2-ylethyl, 1-morpholin-2-ylmethyl, (5-amino-1,3-dioxan-2-yl)ethyl, and (5-amino-1,3-dioxan-2-yl)ethyl.

16. A compound selected from the group consisting of:

| | |
|---|---|
| 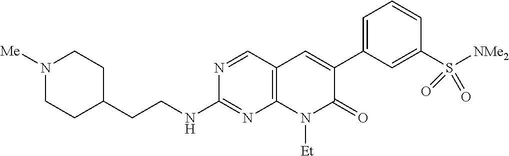 [SMILES: CCn1c(=O)c(-c2cccc(S(=O)(=O)N(C)C)c2)cc2cnc(NCCC3CCN(C)CC3)nc21] | 3-[8-ethyl-2-[2-(1-methyl-4- piperidyl)ethylamino]-7-oxo- pyrido[2,3-d]pyrimidin-6-yl]- N,N-dimethyl- benzenesulfonamide |
| 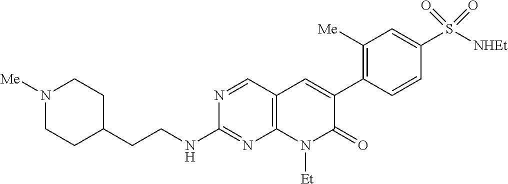 | N-ethyl-4-[8-ethyl-2-[2-(1- methyl-4-piperidyl)ethylamino]- 7-oxo-pyrido[2,3-d]pyrimidin-6- yl]-3-methyl- benzenesulfonamide |
| 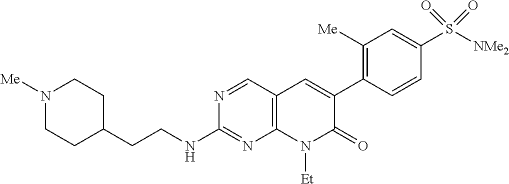 [SMILES: CCn1c(=O)c(-c2ccc(S(=O)(=O)N(C)C)cc2C)cc2cnc(NCCC3CCN(C)CC3)nc21] | 4-[8-ethyl-2-[2-(1-methyl-4- piperidyl)ethylamino]-7-oxo- pyrido[2,3-d]pyrimidin-6-yl]- N,N,3-trimethyl- benzenesulfonamide |
| 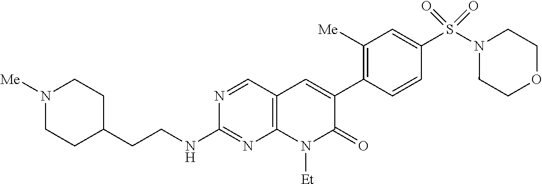 [SMILES: CCn1c(=O)c(-c2ccc(S(=O)(=O)N3CCOCC3)cc2C)cc2cnc(NCCC3CCN(C)CC3)nc21] | 8-ethyl-6-(2-methyl-4- morpholinosulfonyl-phenyl)-2- [2-(1-methyl-4- piperidyl)ethylamino]pyrido[2,3- d]pyrimidin-7-one |
| 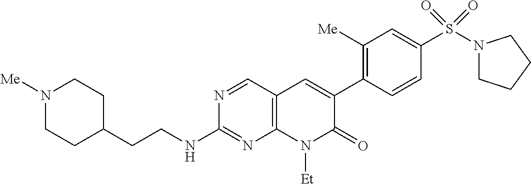 [SMILES: CCn1c(=O)c(-c2ccc(S(=O)(=O)N3CCCC3)cc2C)cc2cnc(NCCC3CCN(C)CC3)nc21] | 8-ethyl-2-[2-(1-methyl-4- piperidyl)ethylamino]-6-(2- methyl-4-pyrrolidin-1- ylsulfonyl-phenyl)pyrido[2,3- d]pyrimidin-7-one |
| 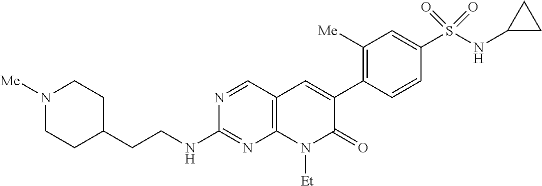 [SMILES: CCn1c(=O)c(-c2ccc(S(=O)(=O)NC3CC3)cc2C)cc2cnc(NCCC3CCN(C)CC3)nc21] | N-cyclopropyl-4-[8-ethyl-2-[2- (1-methyl-4- piperidyl)ethylamino]-7-oxo- pyrido[2,3-d]pyrimidin-6-yl]-3- methyl-benzenesulfonamide |
| 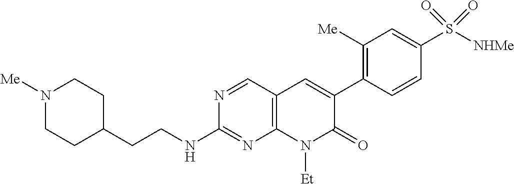 [SMILES: CCn1c(=O)c(-c2ccc(S(=O)(=O)NC)cc2C)cc2cnc(NCCC3CCN(C)CC3)nc21] | 4-[8-ethyl-2-[2-(1-methyl-4- piperidyl)ethylamino]-7-oxo- pyrido[2,3-d]pyrimidin-6-yl]- N,3-dimethyl- benzenesulfonamide |
| 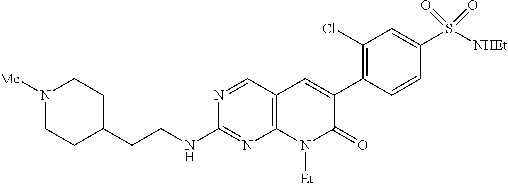 | 3-chloro-N-ethyl-4-[8-ethyl-2- [2-(1-methyl-4- piperidyl)ethylamino]-7-oxo- pyrido[2,3-d]pyrimidin-6- yl]benzenesulfonamide |
| 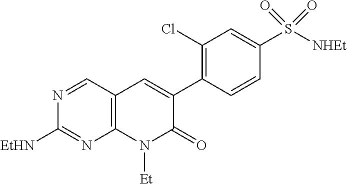 | 3-chloro-N-ethyl-4-[8-ethyl-2- (ethylamino)-7-oxo-pyrido[2,3- d]pyrimidin-6- yl]benzenesulfonamide |
| 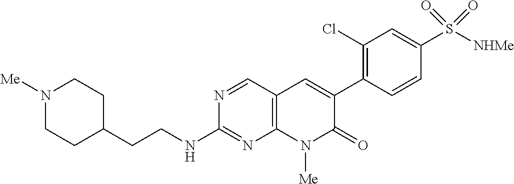 [SMILES: CNS(=O)(=O)c1ccc(-c2cc3cnc(NCCC4CCN(C)CC4)nc3n(C)c2=O)c(Cl)c1] | 3-chloro-N-methyl-4-[8-methyl- 2-[2-(1-methyl-4- piperidyl)ethylamino]-7-oxo- pyrido[2,3-d]pyrimidin-6- yl]benzenesulfonamide |
| 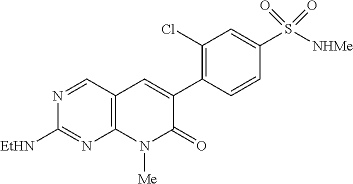 | 3-chloro-4-[2-(ethylamino)-8- methyl-7-oxo-pyrido[2,3- d]pyrimidin-6-yl]-N-methyl- benzenesulfonamide |
| 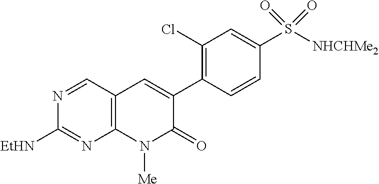 | 3-chloro-4-[2-(ethylamino)-8- methyl-7-oxo-pyrido[2,3- d]pyrimidin-6-yl]-N-isopropyl- benzenesulfonamide |
| 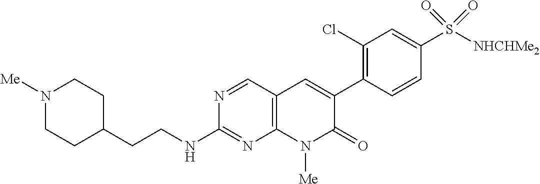 | 3-chloro-N-isopropyl-4-[8- methyl-2-[2-(1-methyl-4- piperidyl)ethylamino]-7-oxo- pyrido[2,3-d]pyrimidin-6- yl]benzenesulfonamide |
| 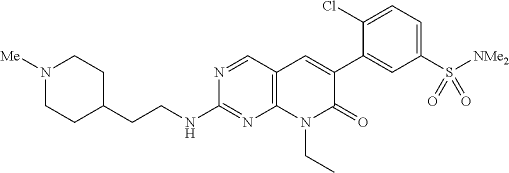 [SMILES: CCn1c(=O)c(-c2cc(S(=O)(=O)N(C)C)ccc2Cl)cc2cnc(NCCC3CCN(C)CC3)nc21] | 4-chloro-3-[8-ethyl-2-[2-(1- methyl-4-piperidyl)ethylamino]- 7-oxo-pyrido[2,3-d]pyrimidin-6- yl]-N,N-dimethyl- benzenesulfonamide |
| 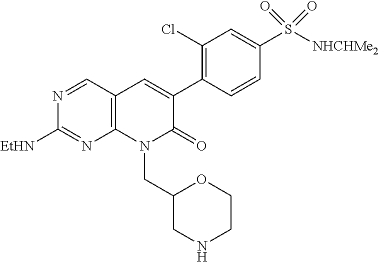 | 3-chloro-4-(2-ethylamino-8- morpholin-2-ylmethyl-7-oxo- 7,8-dihydro-pyrido[2,3- d]pyrimidin-6-yl)-N-isopropyl- benzenesulfonamide |
| 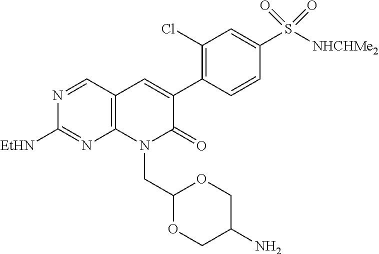 | 4-[8-(5-amino-[1,3]dioxan-2- ylmethyl)-2-ethylamino-7-oxo- 7,8-dihydro-pyrido[2,3- d]pyrimidin-6-yl]-3-chloro-N- isopropyl-benzenesulfonamide |
| 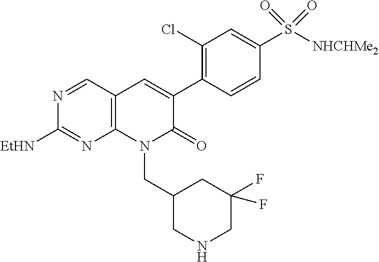 | 3-chloro-4-[8-(4,4-difluoro- piperidin-3-ylmethyl)-2- ethylamino-7-oxo-7,8-dihydro- pyrido[2,3-d]pyrimidin-6-yl]-N- isopropyl-benzenesulfonamide |

and pharmaceutically acceptable salts thereof.

17. A pharmaceutical composition comprising a compound according to claim 1, or a pharmaceutically acceptable salt thereof, and at least one pharmaceutically acceptable carrier, excipient or diluent.

18. A method of treating or ameliorating the severity of cancer or a hyperproliferative disorder in a patient in need thereof comprising administering to said patient an effective amount of a compound according to claim 1, or a pharmaceutically acceptable salt thereof.

19. The method according to claim 18, wherein said cancer or hyperproliferative disorder is selected from the group consisting of adenoma, bladder cancer, brain cancer, breast cancer, colon cancer, epidermal carcinoma, follicular carcinoma, cancer of the genitourinary tract, glioblastoma, Hodgkin's disease, head and neck cancers, heptoma, keratoacanthoma, kidney cancer, large cell carcinoma, leukemias, lung adenocarcinoma, lung cancer, lymphoid disorders, melanoma and non-melanoma skin cancer, myelodysplastic syndrome, neuroblastoma, non-Hodgkins lymphoma, ovarian cancer, papillary carcinoma, pancreatic cancer, prostate cancer, rectal cancer, sarcoma, small cell carcinoma, testicular cancer, tetracarcinomas, thyroid cancer, and undifferentiated carcinoma.

20. The method according to claim 18, wherein said cancer or hyperproliferative disorder is selected from the group consisting of lung cancer, breast cancer, ovarian cancer, bladder cancer, head and neck cancer, primary breast adenocarcinoma, squamous non-small cell lung cancer, and squamous head and neck cancer.

21. The method according to claim 18, wherein said compound of claim 1, or a pharmaceutically acceptable salt thereof, is co-administered with at least one other chemotherapeutic agent used to treat or ameliorate cancer or a hyperproliferative disorder.

22. The method according to claim 21, wherein the other chemotherapeutic agent is selected from the group consisting of inhibitor of apoptosis proteins (IAP), an EGFR inhibitor or antagonist, an inhibitor of Ras/Raf/Mek/Erk signaling cascade, an inhibitor of Akt kinase and a Src kinase inhibitor.
